(19) United States
(12) Patent Application Publication
(10) Pub. No.: US 2024/0136055 A1
(43) Pub. Date: Apr. 25, 2024

(54) A Privacy Enabled System And Method For Managing Logistics For Clinical Study Participants

(71) Applicant: Gray Consulting, Inc., Philadelphia, PA (US)

(72) Inventors: SCOTT GRAY, Fort Lauderdale, FL (US); WILLIAM WING, San Francisco, CA (US); TREVOR SMITH, Salt Lake City, UT (US); BRENT SNYDER, Souderton, PA (US); DONNA BRUMBACK, Raleigh, NC (US)

(21) Appl. No.: 18/278,210

(22) PCT Filed: Nov. 18, 2022
(86) PCT No.: PCT/US2022/050355

§ 371 Date: Aug. 21, 2023

Related U.S. Application Data
(60) Provisional application No. 63/281,046, filed on Nov. 18, 2021.

(51) Int. Cl. G16H 40/20; G16H 10/20

(52) U.S. Cl. CPC G16H 40/20; G16H 10/20

(57) ABSTRACT

Systems for visualization and management of a travel policy for a clinical study have a study dataset having a first travel policy condition, a study travel policy dataset having a main travel policy and a specialized travel policy, and a GUI having a travel policies interface and a travel policy control. The first travel policy condition may be a country, a clinical site, a traveler policy label, a visit attribute, a visit policy label, and combinations thereof. The specialized travel policy (a country policy, a site policy, a first visit policy, a second visit policy, a custom policy, and combinations thereof) is programmatically associated with the first travel policy condition.

### CROSS-REFERENCE TO RELATED APPLICATIONS

This application claims the benefit of U.S. Provisional Patent Application 63/281,046, filed Nov. 18, 2021, and is continuation-in-part of Patent Cooperation Treaty Application PCT/US22/21443, filed Mar. 22, 2022, both of which are hereby incorporated by reference in their entirety.

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### BACKGROUND OF THE INVENTION

The present invention relates to a system for managing participants in a clinical study. Clinical studies may be organized, funded, or sponsored by various entities, or sponsors, (e.g., medical or pharmaceutical companies, government agencies, hospitals, doctors, health care providers, and others). Sponsors may set various requirements for the study, including for example, the time, length, schedules, and goals of the study; the drugs or medications to be studied and their dosages; the treatments or procedures to be studied and their duration; the locations where to perform procedures and tests; the types and numbers of study participants, rules and policies for the management and recruitment of study participant, and various other rules and requirements. Sponsors may also establish the study budgets and financial rules.

Clinical studies can be expensive, with substantial budgets for identification, recruitment, and management of volunteer study participants. When participants drop out of a study, it may lead to substantial additional costs, including due to replacements, delays, duplications, and various other reasons. Therefore, reducing the number of participants who drop out of a clinical study may lead to a reduction, or more efficient use, of study budgets.

Participants may drop out of studies for various reasons, including difficulties traveling to study locations, financial challenges, scheduling interference with work or family commitments, and others. Patients (“participants” and “patients” are used interchangeably below) in a clinical study may have health conditions that constrain their abilities and availability. It is therefore desirable to provide a way of assisting patients by unburdening them and alleviating study participation challenges. One way may be by providing streamlined methods (e.g., apps, software, website, call centers, etc.) that participants can use at their convenience for participation related tasks, such as booking travel, expense reimbursement, and others. Such solutions that may allow participants to, for example, arrange their own travel, still demand time and effort from participants. Also, for privacy, security, consistency, simplicity, and various other reasons, participant-facing systems may need to enforce the same study-wide travel and financial policies for all participants who book their own travel, making it difficult to apply individualized travel policies. Embodiments of the present invention enable an efficient and automated concierge type service allowing travel policy individualization for each participant and reducing participants' time spent managing their participation in the clinical study.

Additionally, systems that collect, store, process, access, or display, personal or protected health information must implement strict privacy, data security, and auditing measures compliant with multi-jurisdictional privacy and data security laws, and regulations. Implementing appropriate security measures while providing individualizing functionality in participant-facing systems could become very complex, considering that such systems, e.g., a mobile app, may be accessed by thousands of individuals, in multiple international jurisdictions, over public networks, and in insecure locations, to name a few potential risks.

Various national and international legal frameworks (e.g., HIPAA and HITECH in the USA; GDPR in the EU, privacy laws in the UK, Russia, Turkey, Canada, Australia, and many others) (“Privacy Laws”) establish complex standards intended to guard various information related to individuals, including Personal Information (PI), personally identifiable information (PII), personal data (PD), protected health information (PHI) (including GDPR's, “Data concerning health” or “Health Records”).

For consistency and ease of reference, below “Personal Information” or “PI” are used to refer to all types of sensitive personal information, including PII, PD, Health Records and PHI, that may be regulated by data privacy law or regulation in any jurisdiction.

Various Privacy Laws provide recommendations, and/or requirements related to the types of security actions used to protect PI, including, by way of example i) use of encoding algorithms and high-security decryption tools; ii) employing pseudonymization and de-identification, iii) testing, assessing, and evaluating the effectiveness of data security measures and the ability to send timely data breach notifications; and iii) employing processes to ensure the ongoing confidentiality, integrity, availability and resilience of data processing systems and services. With respect to encoding/decoding, it may be preferred (or even required) that decryption tools be stored separately from the data they are used to decrypt. Data pseudonymization and de-identification refers to data management techniques and procedures that replace certain data, such as PI, with artificial identifiers, or pseudonyms thereby reducing privacy breach risks. Various Privacy Laws also may require the implementation of auditing capabilities to allow examination of detailed activity logs or reports showing who had access to Personal Information data, from what IP addresses, what data were accessed, and other information

### BRIEF SUMMARY OF THE INVENTION

Embodiments of the present invention are directed to systems for visualization and management of a travel policy for a clinical study. In one embodiment the system comprises a study dataset having a first travel policy condition, a study travel policy dataset comprising a main travel policy and a specialized travel policy, and a GUI. The GUI comprises a travel policies interface and a travel policy control. The first travel policy condition is selected from the group consisting of a country, a clinical site, a traveler policy label, a visit attribute, a visit policy label, and combinations thereof. The specialized travel policy is programmatically associated with the first travel policy condition. The specialized travel policy is selected from a country policy, a site policy, a first visit policy, a second visit policy, a custom policy, and combinations thereof. The GUI receives from the database the travel policy condition and the study travel policy dataset and. The travel policies interface displays the travel policy condition, the main travel policy, and the specialized travel policy. The travel policies interface display is configured to visually associate the first travel policy condition with the specialized travel policy. The GUI is configured to enable using the travel policy control to modify the specialized travel policy. The GUI, responsive to using the travel policy control to modify the specialized travel policy, is configured to receive a modified specialized travel policy, store the modified specialized travel policy in the database replacing the specialized travel policy, cause the database to programmatically associate the modified specialized travel policy with the first travel policy condition, and update the travel policies interface to visually associate the travel policy condition with the modified specialized travel policy.

In another embodiment the GUI is configured to enable using the travel policy control to add a second travel policy condition. The GUI, responsive to using the travel policy control to add the second travel policy condition, is configured to receive the second travel policy condition, store the second travel policy condition in the database, and update the travel policies interface to display the second travel policy condition. The GUI is configured to enable using the travel policy control to add a new specialized travel policy. The GUI, responsive to using the travel policy control to add the new specialized travel policy, is configured to receive the new specialized travel policy, receive information identifying the first travel policy condition or the second travel policy condition, store the new specialized travel policy in the database, cause the database to programmatically associate the new specialized travel policy with either the travel policy condition first or the second travel policy condition according to the information identifying the first travel policy condition or the second travel policy condition, and update the travel policies interface to display the specialized travel policy in visual association with either the first travel policy condition or the second travel policy condition according to the information identifying the first travel policy condition or the second travel policy condition.

In yet another embodiment the main travel policy comprises a main travel rule, and a main expense rule. The specialized travel policy, the modified specialized travel policy, and the new specialized travel policy, each comprise a specialized travel rule, a modified specialized travel rule, and a new specialized travel rule, respectively. The travel policies interface displays the first travel policy condition in visual association with the specialized travel policy and the second travel policy condition in visual association with the new specialized travel policy. The travel policies interface may display the first travel policy condition, the second travel policy condition, the specialized travel policy, and the new specialized travel policy as a table. The first travel policy condition is displayed visually aligned with the specialized travel rule and the specialized expense rule and the second travel policy condition is displayed visually aligned with the new specialized travel rule and the new specialized expense rule.

In another embodiment the clinical dataset comprises a first participant dataset having the first travel policy condition. The specialized travel policy is programmatically associated with the first participant dataset. The clinical dataset comprises a second participant dataset. The GUI comprises a participant travel policies interface having a participant policy control. The participant travel policies interface receives from the database and displays the second participant dataset. The GUI is configured to enable using the participant policy control to associate the second participant dataset with the second travel policy condition. Responsive to using participant policy control the GUI is configured to cause the database to programmatically associate the second travel policy condition with the second participant dataset and cause the database to programmatically associate the new specialized travel policy with the second participant dataset. The second travel policy condition is selected from the country, the site, and the traveler policy label. The new specialized travel policy is selected from the country policy, the site policy, and the custom policy.

In yet another embodiment the clinical study dataset comprises a first visit and a second visit. The first visit comprises a first visit attribute and the second visit comprises a visit policy label. The travel policy data set comprises the first visit policy associated with the first visit attribute, and the second visit policy associated with the visit policy label. The first visit policy is programmatically associated with the first visit and the second visit policy is programmatically associated with the second visit. The GUI comprises a visit schedule control, and the GUI is configured to enable using the visit schedule control to add a third visit having the first visit attribute. Responsive to using the visit schedule control to add the third visit, the GUI is configured to cause the database to programmatically associate the third visit with the first visit policy.

In still another embodiment the clinical study dataset comprises a fourth visit. The GUI is configured to enable using the visit schedule control to associate the fourth visit with the visit policy label. Responsive to using the visit schedule control to associate the fourth visit with the visit policy label, the GUI is configured to cause the database to programmatically associate the fourth visit with the second visit policy.

### BRIEF DESCRIPTION OF THE SEVERAL VIEWS OF THE DRAWING

The advantages and features of the present invention will be better understood as the following description is read in conjunction with the accompanying drawings, wherein:

FIG. 1 is a diagram illustrating an embodiment of the present invention.

FIG. 2 is a diagram illustrating a database embodiment in an embodiment of the present invention.

FIG. 3 is a diagram illustrating a flowchart of an embodiment of the present invention.

FIG. 4 is a diagram illustrating a flowchart of an embodiment of the present invention.

FIG. 5 is a diagram of an embodiment of a clinical study graphical user interface in an embodiment of the present invention.

FIG. 6 is a diagram of an embodiment of a clinical study graphical user interface in an embodiment of the present invention.

FIG. 7 is a diagram of an embodiment of a graphical user interface of an embodiment of the present invention.

FIG. 8 is a diagram of an embodiment of a clinical study graphical user interface in an embodiment of the present invention.

FIG. 9 is a diagram of an embodiment of a clinical study graphical user interface in an embodiment of the present invention.

FIG. 10 is a diagram of an embodiment of a clinical study participant graphical user interface in an embodiment of the present invention.

FIG. 11 is a diagram of an embodiment of a clinical study participant graphical user interface in an embodiment of the present invention.

FIG. 12 is a diagram of an embodiment of a clinical study participant travel profile graphical user interface in an embodiment of the present invention.

FIGS. 13-26 are diagrams illustrating embodiments of travel profiles of a clinical study participant in an embodiment of the present invention.

FIG. 27 is a diagram of an embodiment of a clinical study participant travel graphical user interface in an embodiment of the present invention.

FIG. 28 is a diagram illustrating an embodiment of an expenses interface in an embodiment of the present invention.

FIG. 29 is a diagram of an embodiment of a clinical study sites graphical user interface in an embodiment of the present invention.

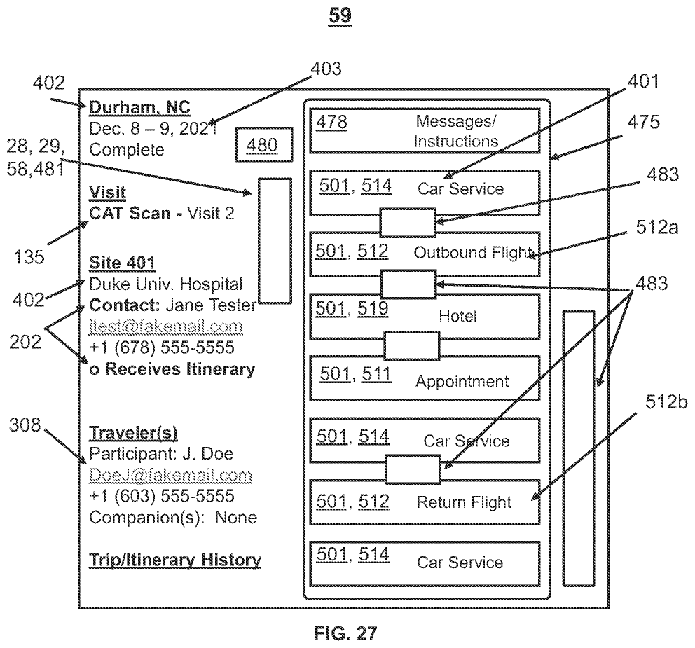

FIG. 30 is a diagram of an embodiment of a clinical study visit schedule graphical user interface in an embodiment of the present invention.

FIG. 31 is a diagram of an embodiment of a clinical study participant visit schedule graphical user interface in an embodiment of the present invention.

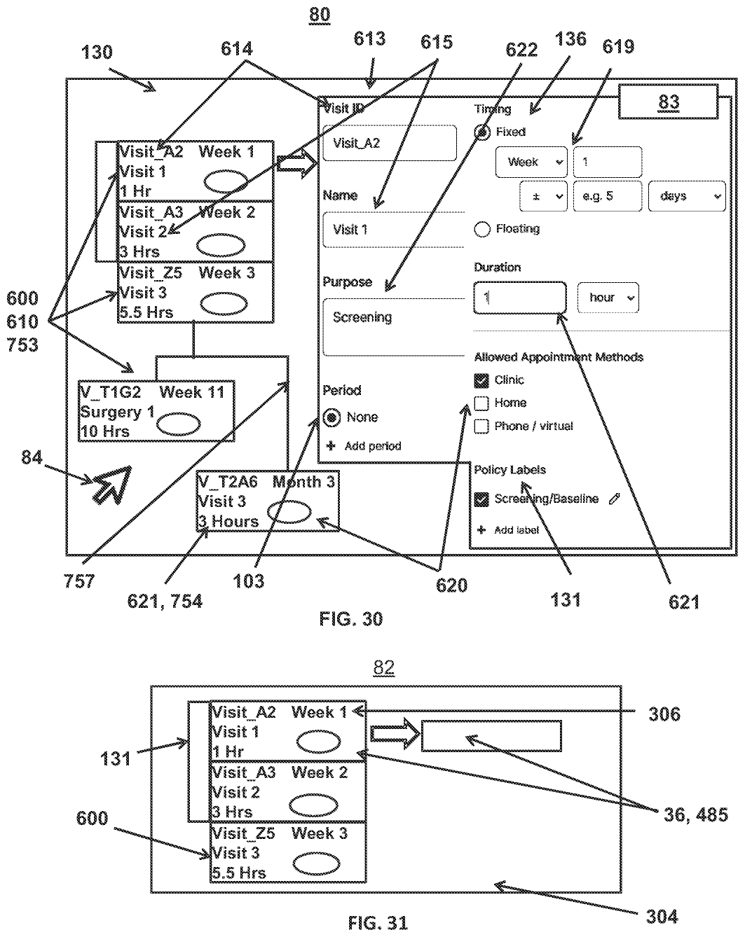

FIGS. 32-35 are diagrams of embodiments of clinical study participant travel graphical user interface in an embodiment of the present invention.

FIG. 36 is a diagram of an embodiment of a travel planning graphical user interface in an embodiment of the present invention.

FIG. 37 is a diagram of an embodiment of a clinical study participant travel policies graphical user interface in an embodiment of the present invention.

FIG. 38 is a diagram of an embodiment of a clinical study travel policies graphical user interface in an embodiment of the present invention.

FIG. 39 is an illustration of an embodiment of a clinical study travel policy in an embodiment of the present invention.

For clarity all reference numerals may not be included in every figure.

### DETAILED DESCRIPTION OF THE INVENTION

The present invention is related to the management of individuals, and associated personal information, participating in a broad range of experimental or observational medical, health, sociological, and other scientific research or studies, collectively referred below as clinical study 25. While the present disclosure refers to clinical studies to illustrate embodiments of the invention, the invention should not be understood to be limited to only clinical studies.

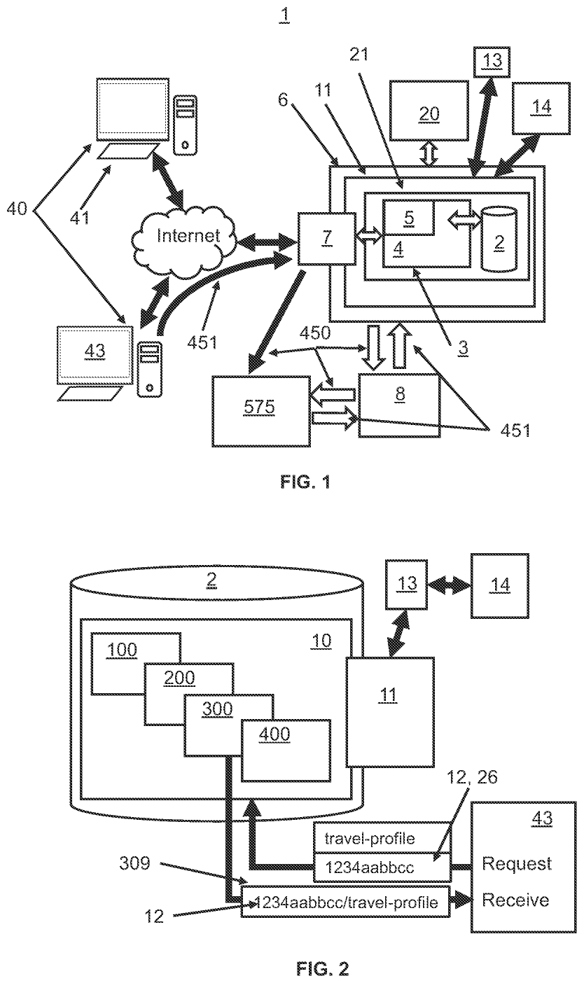

FIG. 1 illustrates a system according to a preferred embodiment of the present invention, which may be implemented as a distributed, networked, system 1 utilizing a database 2, server 3, private cloud 6, and user workstations 40. System 1 may further utilize an internet gateway 7, monitoring tools 20, and data encryption tools 11.

Database 2 may be a SQL, Non-SQL, relational, or non-relational database (e.g., MySQL, Oracle, Mongo, Cassandra, ElasticSearch, Neo4J, and others) or any other datastore or repository for persistently storing and managing collections of data according to the invention. Database 2 may comprise a database server comprising a processor. Preferably, database 2 is secured within the private cloud 6, and may be further secured by isolating it in a private subnet within the private cloud 6, effectively preventing anyone outside the private cloud from gaining access to database 2. All data stored in database 2 preferably are encrypted while at rest (e.g., when stored in database 2) using encryption tools 11.

Data 10 stored in database 2 preferably are further protected, and/or obfuscated, for example by associating data records with randomly generated unique identifiers 12 (e.g., uuid, guid, others) and using the unique identifiers 12 to query, access, and/or reference records or portions of data 10. Unique identifiers 12 allow data 10 to be de-identified or pseudonymized. For example, in a SQL database 2, unique identifier 12 may be used as a primary table key, or in a lookup table associating unique identifier 12 with data or even a datum. For non-SQL/non-relational database 2, the unique identifier may be used to access records or values in, for example, as a key or document identifier, within nodes/vertices, in key-value pairs, and other methods that use unique identifier 12 instead of referencing actual data values. Other methods of using unique identifier 12 and other methods of protecting data in relational and non-relational databases are well known. Servers 3 preferably are secured within private cloud 6 and may be web servers 4, application servers 5, or both, and may use dedicated or shared computing resources.

Workstation 40 may use display 42 to display data received from database 2 or from input device 41. Workstation 40 may comprise a graphical user interface (GUI) 43 that visualizes and facilitates the input and manipulation of data by a user using workstation 40 and input device 41. A user may be any person authorized to access system 1 through a workstation 40, and who may be associated with a study, such as staff affiliated with study sponsors, CROs, clinical sites, institutions, vendors, participants, and others. Workstation 40 may be any computing device comprising a processor, such as a personal computer, laptop, tablet, mobile device, thin client, or any other device capable of displaying the GUI and connecting to a network (e.g., the internet, internal networks, other public or private networks). Input device 41 may be a mouse, keyboard, microphone, voice, optical input, camera, tape, and other known devices or methods for inputting data.

Display 42 may be any display or monitor (e.g., screen, projector, e-ink, holographic, etc.) and a display controller (e.g., display hardware and software controlling the display), as well as any other hardware or software instrumentality, or interface used or known to properly operate display 42. Display 42 may display GUI 43 using client software or program capable of visualizing information received over a network and/or input device 41, and of securely transmitting the information over a network, for example, a browser capable of utilizing network protocols (e.g., HTTP, HTTPS, AS2, AS3, AS4, RTP, UDP, etc.) to send and receive data over networks and of displaying data using markup (e.g., HTML, XML, SGML, etc.) or other visualization techniques. In embodiments with more than one workstation 40, each display 42 may display different data, and different aspects and/or views of GUI 43 permitting different users to simultaneously perform different actions.

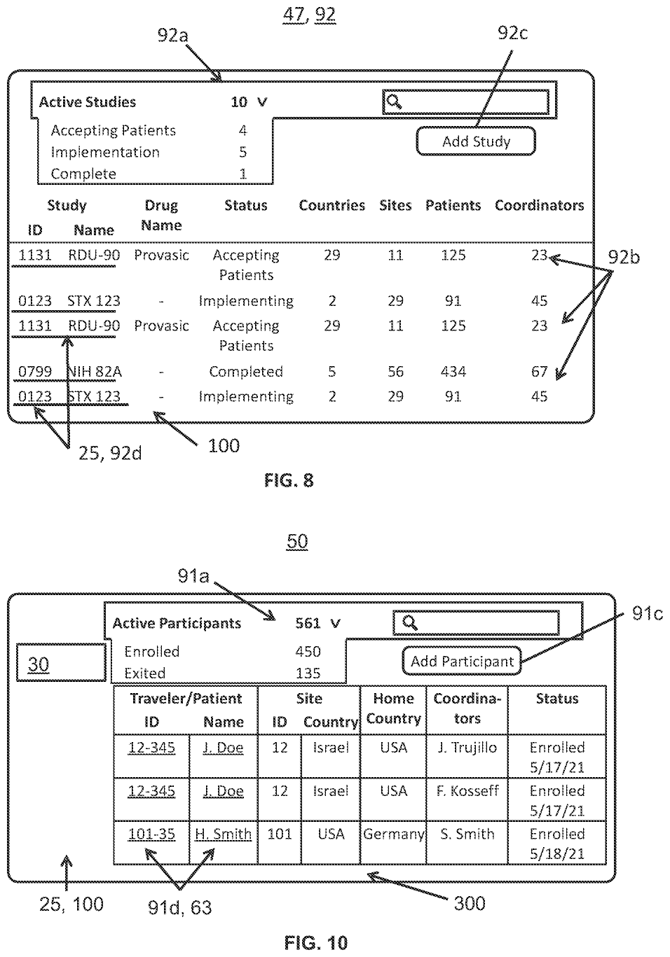

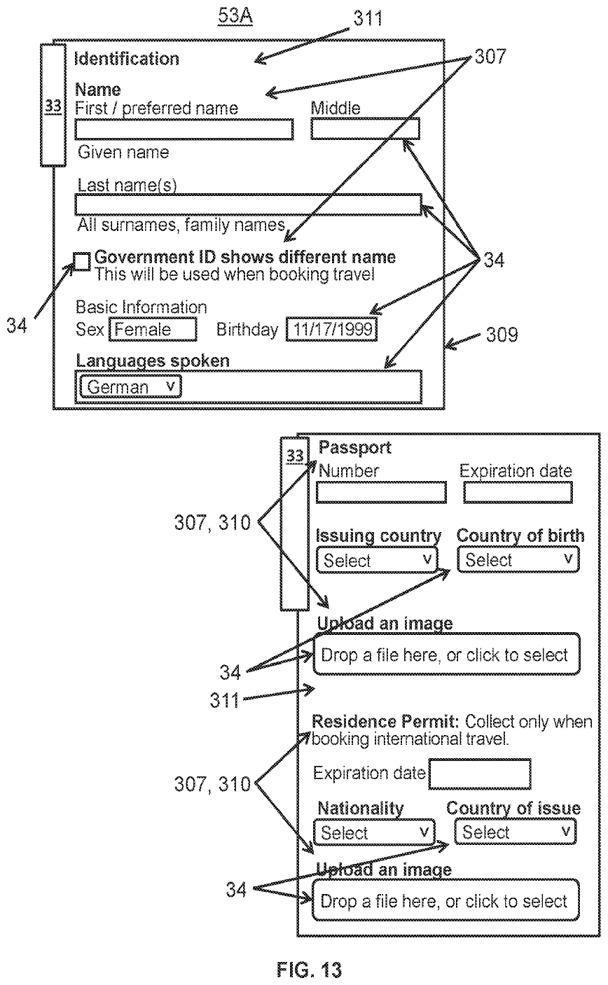

An embodiment of GUI 43 illustrated in FIG. 7, facilitates the efficient input and customization of data as well as efficient navigation through multiple visual panels and views of data 10, configured to efficiently facilitate different operations, such as entry, modification, and visualization of participant data, clinical site data, travel policies data, user security assignments, visit schedules, travel booking and travel management (including transportation and accommodations), expense management and reimbursement, and various others. The graphical user interface 43 comprises a control panel 44, main search control 45, and interface 47. In one embodiment, GUI 43 is configured to enable a user to utilize control panel 44 and controls 44a to access interface 47. Embodiments of interface 47 illustrated in FIGS. 5-9, may comprise various views and/or panels, including, trips 90, travelers 91, available studies 92, study 98, available sites 93, site 99, coordinators 94, institutions 95, available countries 96, and country 97.

FIG. 7 illustrates an embodiment of GUI 43, control panel 44, main search control 45, and interface 47 are configured to enable efficient navigation between the different panels, views, and interfaces by utilizing (e.g., selecting, clicking, touching, pointing, typing, etc.) various controls (e.g., icons, tab, button, pull down list, menu, search box, clickable link, etc.). For example, GUI 43 may be configured to so that the assignments interface may be accessed (e.g., displayed and switched to) by utilizing an assignments control, participants interface 50 may be accessed with control 51, study sites interface 60 may be accessed with control 61, coordinators interface 94 may be accessed with control 94a, travel policies interface 70 may be accessed with control 71, and visit schedule interface 80 may be accessed by utilizing visit schedule navigation control 81.

Interface 47 may be configured to display various interactive views and panels to permit a user to efficiently provide services for participants, including selecting and booking travel itineraries (e.g., flights, ground transportation, hotels, housing), and providing notes, instructions, and directions for participants and/or travel service providers. Interface 47 of GUI 43 also may be configured to enable input, customization, and/or correction of participant dataset 300, to enter new information, correct existing information, or modify and supplement participant dataset 300 to reflect individualized requirements and preferences of a participant, for example enabling a participant specific travel policies if warranted. GUI 43 may be configured to enable such input, customization, or correction of participant dataset 300 during the provision of concierge-type services by a user/coordinator of system 1, or independently of the provision of such services, to enter new study participants 26.

A trips view 90, exemplified in FIG. 5, may receive from database 2 information from trip dataset 400 about trips associated with one or more studies and displays it, including for example, trip status (occurred, submitted, in progress, upcoming, booking requested, etc.), travelers and itinerary (e.g., traveler name, dates, responsible user, origin, destination), and other information. Trips view 90 may be configured to only display information from trip dataset 400 associated with travelers that a logged-in user is authorized to view, for example if a concierge-user accesses trips view 90, GUI 43 will only receive and display trip data 400 for trips associated with participants 26 who are assigned to the concierge-user. Another user (e.g., a system administrator, manager, etc.) who is permitted to view all participants in data 10, may be able to view the trips associated with all participants. Trips view 90 interface may provide data filter controls 90a, 90b, 90c to filter the displayed information from trip dataset 400 by, for example, trip status, a clinical site or study, specific trip or participant, and by any other data (e.g., by typing a search term). GUI 43 may be configured to enable a user to utilize control 90c associated with a traveler or trip to switch to another interface, for example, participant interface 52. If trip view 90 is filtered by a participant, it may be displayed as participant trips module 56, and may display a travel control 485 allowing the planning of a new trip for that participant 26.

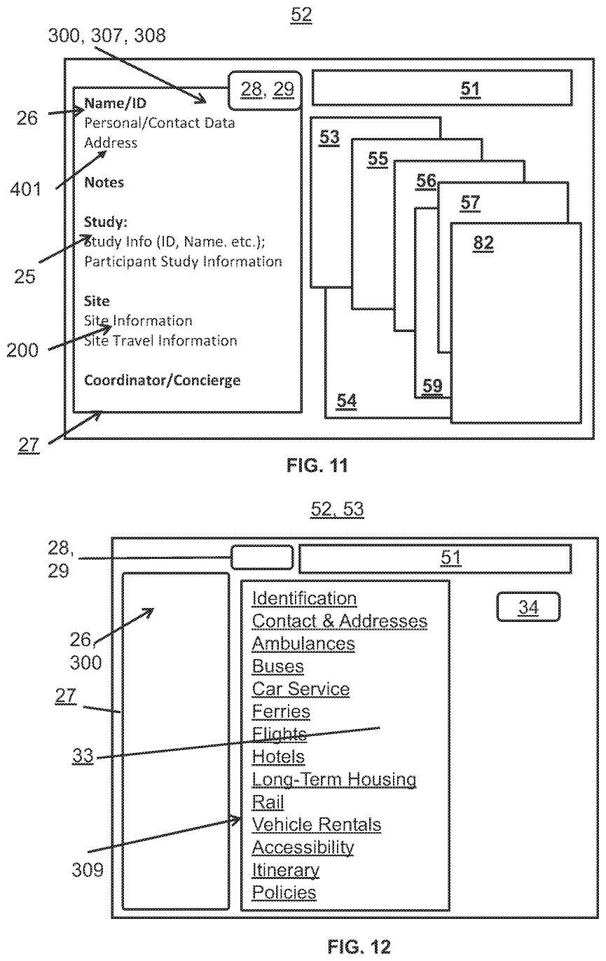

FIG. 6 illustrates an example of travelers view 91, which may receive (e.g., from database 2) and displays study participant data 300, and may also provide controls 91a, 91b to filter the display of travelers view 91 by a specific traveler/participant (traveler, participant, and patient are used interchangeably for clinical study participants). Travelers view 91 may also provide controls 91c (e.g., [Add Participant] button), allowing a user to enter information about a new traveler, or 91d (e.g., a link, url, associated with a participants) allowing a user to switch to participant interface 52. If travelers view 91 is filtered by a study 25 it may be displayed as study participants interface 50 travelers view 91 and study participants interface 50 may be configured to only display participant dataset 300 that a user currently accessing GUI 43 (e.g., logged-in) is assigned to, or authorized to view.

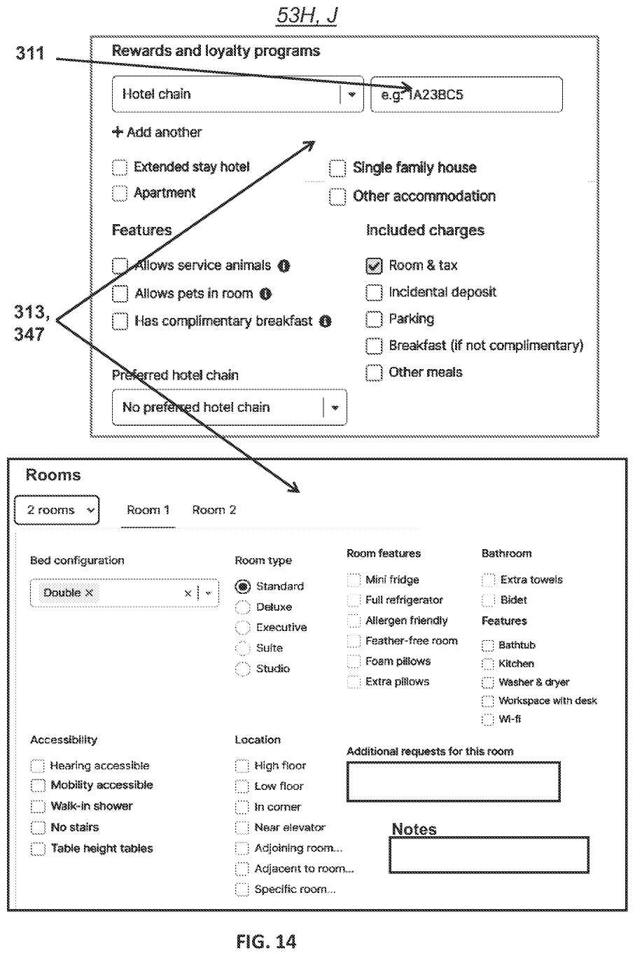

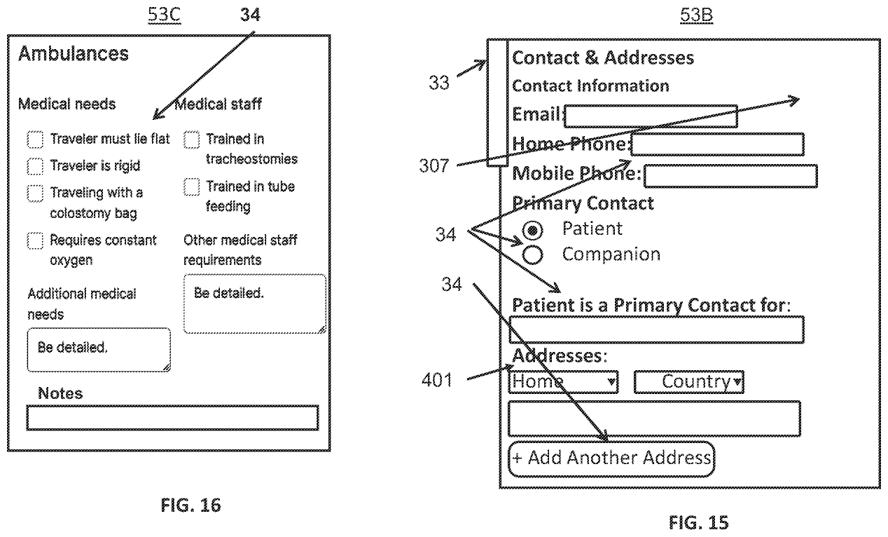

Studies view 92, as exemplified in FIG. 8, receives and displays clinical study dataset 100 data for one or more studies 25, and may provide controls 92a, 92b, 92c, 92d to filter the displayed data by various parameters (e.g., country, type, etc.), add a new study (e.g., [Add study] button), or switch to the study interface 98 (e.g., utilizing control 92d associated with a study) exemplified in FIG. 9.

Study interface receives clinical study dataset 100 for a clinical study 25 from database 2. Study interface 98 may comprise a study summary area 30 displaying summary information from clinical study dataset 100. Study interface 98 comprises one or more interface modules/panels, having data views, interactive panels, and/or interfaces, related to clinical study 25, including, study overview (not shown), assignments interface (not shown), study participants interface 50, participant interface 52, study sites interface 60, travel policies interface 70, visit schedule interface 80, study coordinators interface (not shown), and others. GUI 43 may be configured to provide navigation controls 31 (e.g., tabs, links, button, lists, menu items, radio controls, search boxes, and others) within study interface 98 allowing a user to utilize the navigation controls 31 to switch to different views or panels of study interface 98, or interface 47, to view, input, or manipulate data about clinical study 25, and manage participants 26.

Study overview may be configured to display summary and overview information (e.g., automatically generated and/or aggregated from data 10, or clinical study dataset 100) related to a clinical study 25. Assignments interface may display a list of users with access to the system and their assignments (e.g., levels of access; assigned sites or participants) based on clinical study dataset 100. For example, in some embodiments, assignments may display a list of users who may be coordinators (interchangeably referred to here as users, coordinators, or travel coordinators) tasked to provide concierge-type services, and participants who are assigned to each of those coordinators. A user coordinator may be assigned to participants based on various factors, for example participants associated with a clinical site, participants residing or visiting a particular country, or participants whose language the coordinator speaks. A coordinator may also be assigned a single participant 26, so that a system according to the present invention will permit the coordinator to only access participant dataset 300 of that single participant 26, and no one else's participant dataset 300.

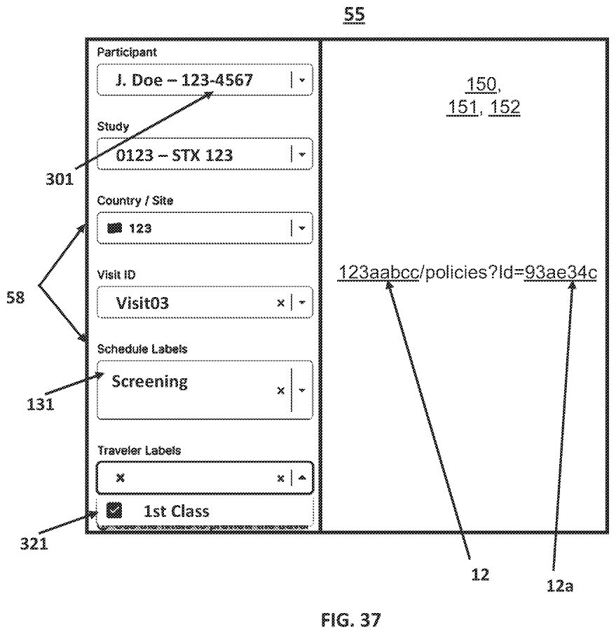

FIG. 38 illustrates an embodiment of a travel policies interface 70 that may receive data about study travel policy 150 for clinical study 25 from database 2, from input device 41, through direct data transmission, and other methods, and may display the study travel policy 150. Travel policies interface 70 may be configured to enable a user to utilize various travel rules controls 159 (also referred to as travel policy controls 159) to modify and customize the study travel policy 150 data so that different travel policies apply to different types of travel or travel circumstances 157 (for simplicity, different types or aspects of travel, and travel circumstances are collectively referred to as “travel policy conditions” 157). Embodiments of travel policies interface 70 may be configured to require an approval (from, e.g., manager) to create and/or customize a specialized travel policy 152, traveler policy labels 321, and/or visit policy labels 131a. If approval is required, additions of and/or changes to a specialized travel policy 152, a traveler policy label 321, and a visit policy label 131 may occur after approval is granted, which may be granted once or on a per need basis.

For example, travel policy interface 70 may provide a travel policy control 159 that enables a user to add new, import, preview, customize, add to, or modify, a study travel policy 150, including a main study travel policy 151, and/or a specialized travel policy 152 and store them in database 2. Travel policy control 159 may further enable a user to create and store in database 2 a traveler label 321 and a visit policy label 131 associated with a specialized travel policy 152. Control 159 may be utilized to create other types of policy labels associated with specialized travel policies 152, such as country policy labels, site policy labels, site location policy labels, and various other travel endpoints, or circumstances.

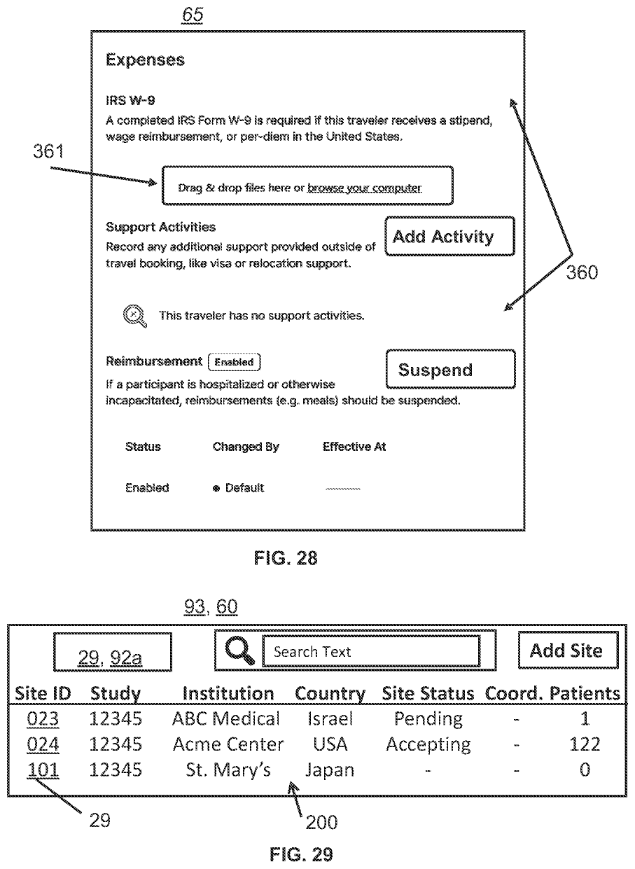

FIG. 30 illustrates an embodiment of a visit schedule interface 80 that receives (e.g., from database 2, input device, data transmission, etc.) and displays study visit schedule 130 data. Visit schedule interface 80 may be configured to enable input, correction, or customization of study visit schedule 130 data utilizing input device 41. Visit schedule interface 80 may provide visit schedule controls 83, 84 that are configured to modify study visit schedule 130 data by selecting, adding, or modifying information about, visits, visit tracks (e.g., clinical protocol arms or patient groups, visually represented as a branch or track in the visit sequence), visit cycles (e.g., repeating one or more visits until a condition is met), visit jumps (e.g., if conditions are met a participant may omit or repeat visits, switch arms/groups, and in general switch to another point in the visit schedule as defined by the configuration of the jump node), information (e.g., about a visit, group of visits, the visit schedule), and others. Visit schedule interface 80 may also provide visit schedule controls 83a to enable a user to add a visit policy label 131 to study visit schedule 130. Visit policy label 131 may be associated with one or more visits, tracks, cycles, and other visit schedule attributes 613 enabling customizable travel policies.

Participant visit schedule module 82 receives (e.g., from database 2, input device, data transmission, etc.) and displays participant visit data 304 for a participant 26. In a preferred embodiment, visit schedule interface 82 may be configured to provide a participant visit control 36 and participant travel control 485 associated with a current visit 306 from participant visit data 304. A user may utilize control 36 to skip a current visit 306, and control 485 to begin planning a new trip and switch to travel interface 500. Participant visit control 36 may also be associated with other visits from participant visit data 304, in which case a user may utilize control 36 to fast forward to that visit, or to make that visit a current visit 306.

Study visit schedule 130 is a dataset comprising data about a clinical participant clinical study visit, also referred to as an appointment, during which a clinical participant may be examined or assessed as part of a clinical study 25. Data in study visit schedule 130 may comprise a visit id 614, visit name 615, visit number, an appointment procedure 135, an appointment schedule 136, visit duration 621 (e.g., associated with the visit name, visit number, and/or appointment procedure 135), and exit strategy. Appointment procedure 135 may be associated with, or it may be based on, one or more of the visit ID 614, visit name 615, visit number, visit tracks, and other visit attributes 613. Appointment schedule 136 may indicate a specific date for a visit, a visit time window 619 with a range of dates (e.g., specific date range; a date range relative to a previous visit, relative to the beginning or end of a study, or relative to another prior or later event or date). Appointment schedule 136 may also indicate a “floating” appointment schedule 136 indicating that the visit (or appointment) can be scheduled at any available time. Appointment schedule 136 may also comprise an appointment start date and time, an appointment end date and time, and/or appointment duration 621. Appointment schedule 136 may also comprise an arrive by date and time 137 and a depart after date and time 138. Arrive by date and time 137 may be based on an appointment start date and time, the appointment procedure 135, and/or other factors, allowing a patient to settle mentally or physically prior to an appointment, to complete pre-appointment preparations, or to accommodate patients with medical or health issues. Depart after date and time 138 may be based on an appointment end date and time, the appointment procedure 135, and/or other factors, allowing a patient time after an appointment procedure 135 for observation, calming, follow up, accommodation for health or medical issues, and others.

Visit id 614 may be an alphanumeric identifier for a visit in a study visit schedule 130. For example, it may be an abbreviation reflecting one or more of a visit name 615, visit number, visit description 622, appointment procedure 135, and other visit data attributes 613. Visit name 615 may be a descriptive name for each visit. Visit name 615 may be the name of a visit from an FDA approved study protocol.

Exit strategy may describe how a patient has exited (e.g., due to an Exit Reason 710), or will exit (e.g., rollover to another study if no early exit), a study. For example, a study visit schedule may comprise multiple exit tracks that allow for participants to exit a study based on different Exit reasons 710 if participants are in different circumstances. A participant who completes a visit schedule may have an end of study (EOS) visit following which the participant may exit the study. Other patients may exit one study and enter another, such as a long-term extension or open label extension study. A patient may also exit a study for various exit reasons 710 (e.g., by choice, adverse effects, illness, death). An exit strategy may be configured depending on the method of exit (e.g., study completion, rollover, early exit reason 710) to move the patient to one of several exit tracks, which may comprise different visits. In an example, early exit due to adverse event exit reason 710 may require an exit track with numerous safety follow-up visits, whereas a patient who completes the entire visit schedule may just attend an end of study visit.

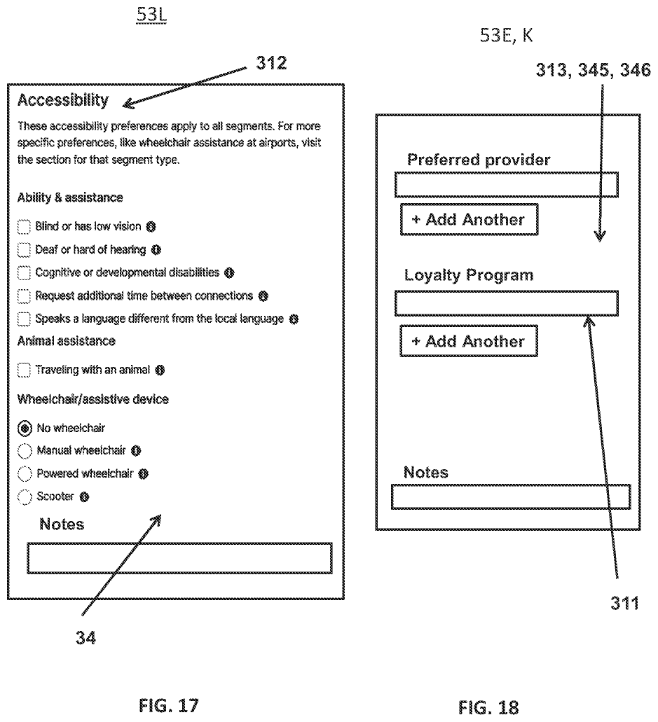

A study participants interface 50, as illustrated in FIG. 10, may display a list of participants in a study who have opted to use the services facilitated by a system according to the present invention, such as a concierge-like travel booking, expense reimbursements, and others. Study participants interface 50 may be configured to display a participant interface 52 by utilizing a control 63 associated with a participant 26 (e.g., clicking on a participant). Participant interface 52 may also be accessed by utilizing the control panel 44, or the main search control 45.

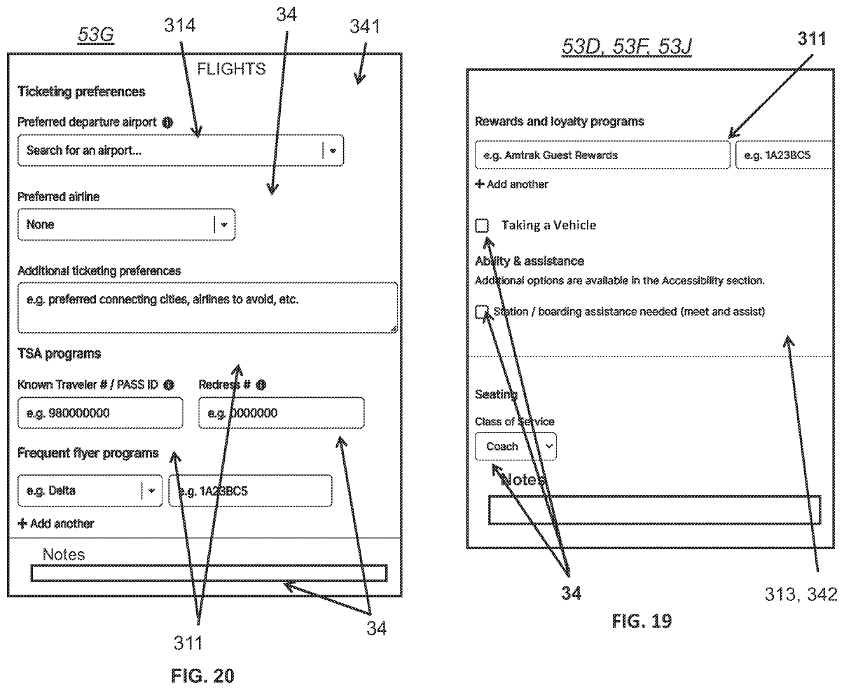

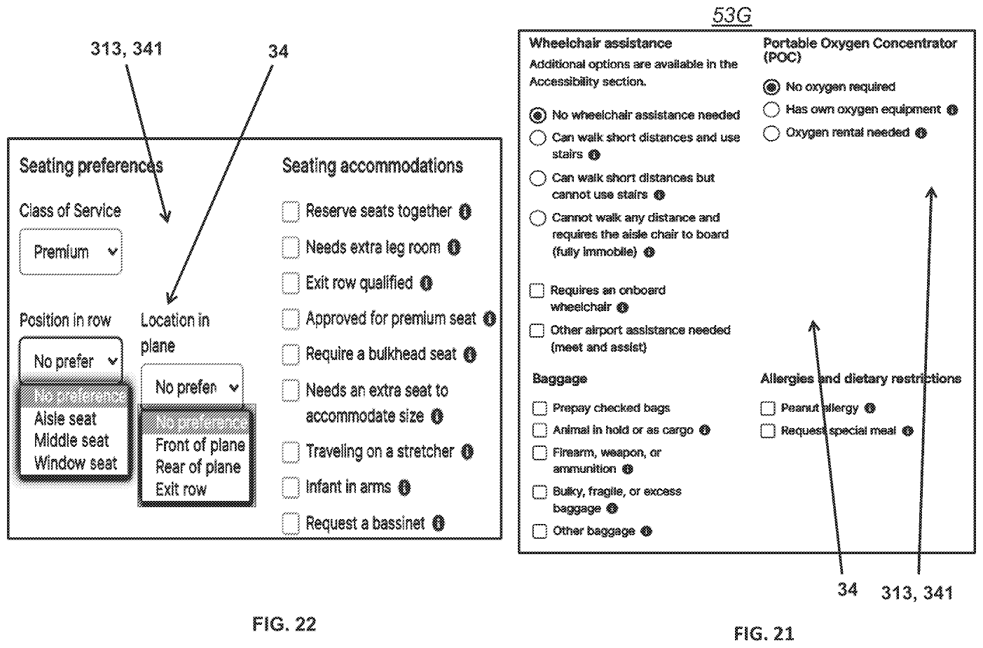

Participant interface 52, exemplified in FIGS. 11, 12, may receive participant dataset 300 about participant 26 from one or more of database 2, input device 41, network data transmission and other methods, and may display participant dataset 300. Participant interface 52 may comprise a summary area 27 displaying information from participant dataset 300. Participant interface 52 may comprise participant data controls 28 configured to enable a user to input, correct, and customize participant dataset-300, by utilizing controls 28 and/or input device 41. For example, by utilizing control 28, a user may enter, or modify participant id, participant site, notes concerning participant 26, participant enrollment date, and may also rollover participant 26 from on clinical study 26 to another clinical study 26 (e.g., an extension study), may exit a participant from a study, remove or add a participant as travel companion (exemplified in FIG. 26), and various other functions. Participant interface 52 may also be configured to allow a user to utilize a site data controls 29, and coordinator controls (not shown) directly from participant interface 52. Participant interface 52 may comprise one or more interface modules having one or more of interactive data views, panels, and interfaces, including travel profile module 53, companion module 54, participant travel policies module 55, participant trips module 56, participant consent interface 57, participant visit schedule module 82, and participant expenses interface 65, each of which may be displayed by the GUI responsive to a user utilizing a participant control 51.

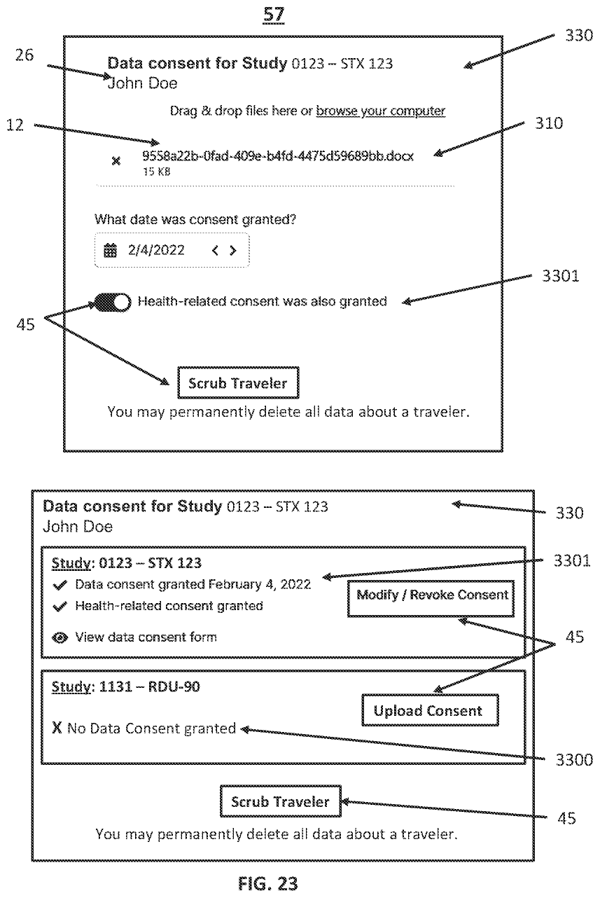

Travel profile module 53, exemplified in FIGS. 12-26, receives traveler profile 309 data from database 2, and provides controls to input, change, modify or customize traveler profile 309. Travel profile module 53 may be viewed in a identification view 53a, contact & addresses view 53b, ambulances view 53c, buses view 53d, car service view 53e, ferries view 53f, flights view 53g, hotels view 53h, long-term housing view 53i, rail view 53j, vehicle rentals view 53k, accessibility view 53l, itinerary view 53m, policies view 53n, each of which may be configured to display, and enable input and modification of, traveler profile data 309. Travel profile module 53 may comprise traveler profile navigation control 33 allowing a user to utilize it to switch one of the travel profile views 53a-53n, and a traveler profile control 34 allowing a user to utilize it to input, modify, or manipulate traveler profile data 309. Traveler profile navigation control 33 may also be configured to allow a user to utilize it to switch any other interface 47, or to any view, module, or interface within participant interface 52, including companion module 54, participant travel policies module 55, participant trips module 56, participant consent interface 57, participant visit schedule module 82, and participant expenses interface 65.

Travel profile module 53 may also be configured to transmit traveler profile 309 to be stored in database 2. Companion module 54, receives companion dataset 325 from database 2, and provides controls to input, change, modify or customize companion dataset 325. Companion module 54 may also be configured to transmit companion data to be stored in database 2.

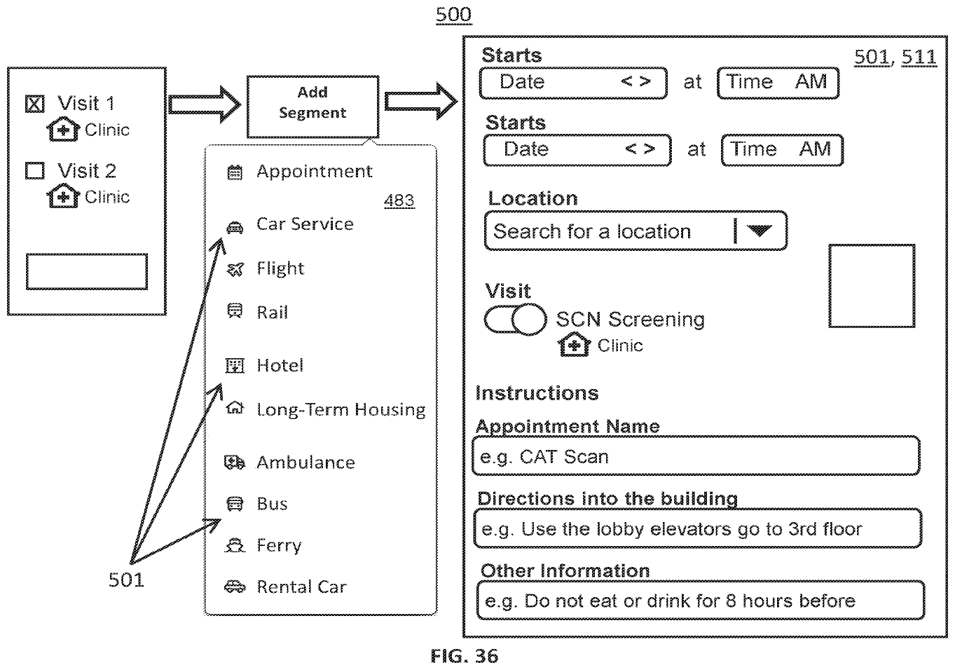

Participant travel policies module 55, exemplified in FIG. 37, enables viewing and customization of participant data 300 related to study travel policy 150 applicable to a participant 26 for a clinical site visit trip, based on main travel policy 151 and any applicable specialized travel policies 152. Participant travel policies module 55 may be configured to display study travel policies 150, including the main study travel policy 151, and any specialized travel policies 152 associated with participant 26 and applicable to participant 26 trips. Participant travel policies module 55 may be configured to allow a participant to be associated or dissociated with a specialized travel policy 152 by utilizing participant policy controls 58 to add or remove an association of participant 26 with a traveler policy label 321 and/or a visit policy label 131 (i.e., a travel policy condition 157). Embodiments may be configured to require an approval (from, e.g., manager) to create and/or customize a specialized travel policy 152, traveler policy labels 321, and/or visit policy labels 131a. If such approval is required, specialized travel policy 152, traveler policy labels 321, and visit policy labels 131 additions and changes may occur after approval is granted.

Participant trips module 56 may display information about past and upcoming participant trips, based on data from a trip dataset 400 and a participant dataset 300 received from database 2. Participant trips module 56 may display information identifying a participant who is taking the trip, a trip status (e.g., pending, submitted, requested, booked, confirmed, etc.), a trip destination, method of transportation, trip schedule, and other information related to a participant trip. Participant trips module 56 may be configured to provide travel control 485 that enables a user to switch to the travel planning interface 500 (e.g., to plan a new trip, modify existing one), or clickable link control 90b embedded in an individual trip text enabling a user to display an individual trip 59 module.

Individual trip 59 module may be configured to receive from database 2, and display, itinerary 475 data, and also may be configured to enable modification of itinerary 475, for example trip details 476, trip planning history 477, trip segment data 501, itinerary message 478, and other information. Individual trip 59 module may also provide controls enabling a user to switch to other interface modules of GUI 43 and to enter and/or modify various data. For example, a user may utilize control 480 to cancel a trip, or to request booking of trip segments by transmitting to booking module 575 data necessary for travel booking, such as booking request data 451. Control 480 may also be utilized to send itinerary 475 to one or more recipients according to traveler contact information 308 and/or the site contact information 202, by utilizing transmission methods and transmission destinations, or recipients, provided in traveler contact information 308 and/or the site contact information 202. For example, itinerary 475 may be sent by emailing it to an email address provided in traveler contact information 308 and/or site contact information 202, or may be sent by facsimile if so indicated in traveler contact information 308 and/or site contact information 202. Traveler contact information 308 and/or site contact information 202 may also indicate that itinerary 475 and other information may be sent by transmitting over a network connection (using, e.g., API calls, secure data/file transfer protocols, and other methods) to a computing system capable of receiving itinerary 475. Such a computing system may be a system associated with a clinical site system, a hospital or another facility, with a patient (e.g., mobile device), and others. Embodiments of the invention may be configured to enable secure online viewing and/or downloading of itinerary 475 or other information by providing a secured portal accessible by authorized persons, for example a contact in a traveler contact information 308 and/or in a site contact information 202, who may be notified (according to the contact information) to log in to the portal. For privacy and security reasons, control 480 may be configured to only allow itinerary 475 to be sent to pre-authorized recipients provided in traveler contact information 308 and site contact information 202.

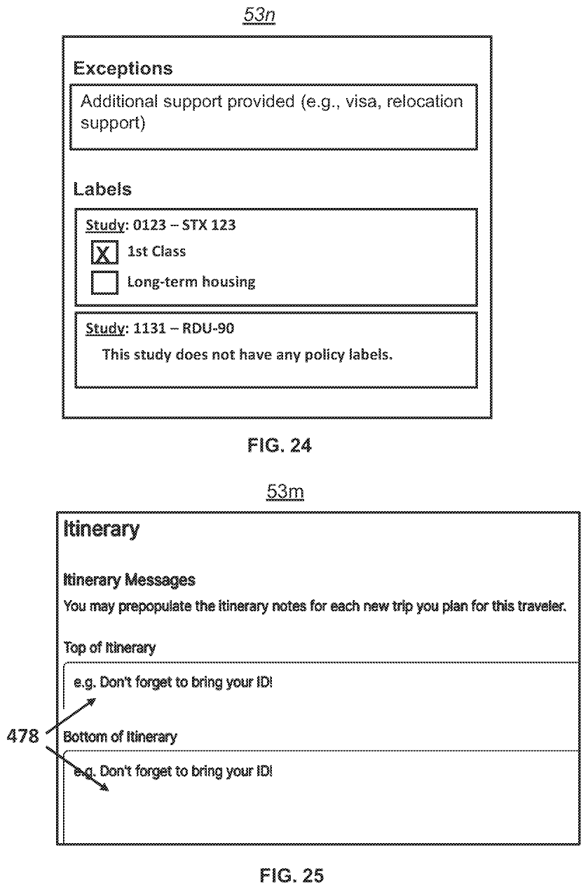

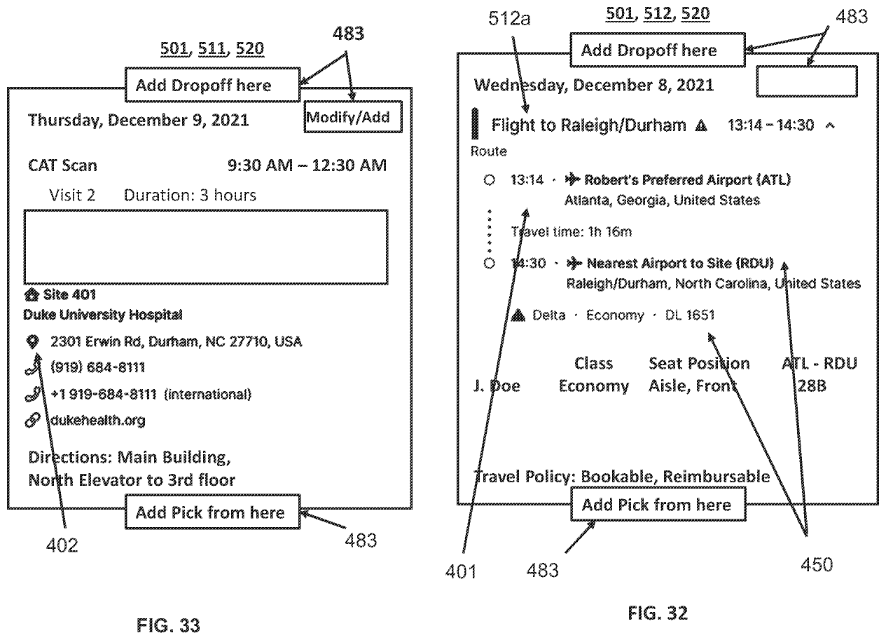

An individual trip 59, exemplified in FIG. 27, module may also be configured to provide controls 481 enabling a user to modify a traveler policy label 321 associated with participant 26 or to add/remove travelers (such as travel companions) to itinerary 475. Individual trip 59 module may also be configured to provide controls 483 to add or modify trip segments by causing the GUI to load the travel planning interface 500, or trip segment panels 520 as exemplified in FIGS. 32-36. For example, control 483 may be associated with a first trip segment start location 503 or with a first trip segment end location 504 of a first (e.g., existing) trip segment data 501, so that a user may utilize control 483 to add a second (e.g., new) trip segment 501 having a second segment start location 503 or a second segment end location 504 based, respectively, on the first trip segment end location 504 or the first trip segment start location 503 of the first (e.g., existing) trip segment data 501. For example, if control 483 is associated with a first trip segment start location 503 at a departure airport, a user may utilize control 483 to add a second car service trip segment 514 having a second trip segment end location 504 be the departure airport location.

FIG. 23 illustrates an embodiment of a participant consent interface 57 receives from the database 2, and displays participant consent record 330, and may also provide consent controls 45 (e.g., slider, checkbox, button, upload controls) configured to allow modification of participant consent record 330. For example, a user may utilize a consent control 45 to indicate whether participant 26 has provided informed consent for the use of personal information, including health information. Consent control 45 may also be used to upload signed personal information data consent forms, for including health records. Consent control 45 may also be configured to initiate a scrubbing (e.g., indicated as “scrub traveler”), or removal, of personal information and health records data of a participant 26, or of all data of a participant 26, from system 1.

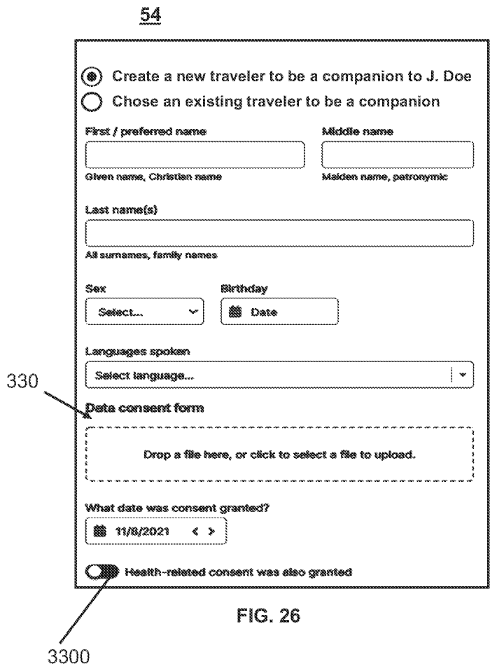

Participant expenses interface 65, exemplified in FIG. 28, receives from the database 2, and displays participant expense data 360, and may also provide expense controls allowing a user to modify and change participant expense data 360. For example, a user may utilize the expense controls to receive (e.g., import for a computer or over a network, drag and drop, etc.) participant tax data 361 (e.g., Tax ID, SSN; IRS forms), indicate support activities provided to a participant 26, add or change status of expenses, and expense rules and policies (e.g., whether reimbursable, expense limits). GUI 43 may also be configured to enable check or validation of tax information (may be initiated automatically or by a user) received for example, from Participant Expenses Interface 65, a W9 form, or elsewhere. In some embodiments check or validation of tax information may involve utilizing the US Internal Revenue Service (IRS) Taxpayer Identification Number (TIN) Matching Tools, by for example, providing, transmitting, and/or querying tax information to an IRS TIN Matching Tool and receiving from the IRS TIN Matching Tool information about the tax information, for example, TIN validation information.

Sites view 93 receives from database 2, and displays, clinical site datasets 200 for clinical sites associated with one or more clinical studies 25. Sites view 93 may provide controls to filter the displayed data by various parameters (e.g., by study, country, search text, and others), for example by study 25, and display site view 93 as study sites interface 60. Site controls may also be utilized to switch to study sites interface 60 or site interface 99, and to enable other functionality (e.g., “add site”).

Study sites interface 60 receives and displays clinical site dataset 200 for sites associated with a clinical study 25. Study sites interface 60 is configured to enable a user to input, correct, or customize clinical site dataset 200 by utilizing a site control 29 and/or input device 41.

Site interface 99 may comprise one or more data views, interactive panels and/or interfaces, including, site participants module (e.g., study participants associated with a site), site assignments module (e.g., users, concierge-users, and other persons assigned to study participants associated with a site), site travel policies module (e.g., travel policies associated with a site, including site labels, visit labels for visits at the site, and traveler labels for participants associated with the site), and others. The site interface may provide a site control 29 allowing the addition of a new clinical site, and modification of clinical site data 200.

Coordinators view 94 receives and displays data about users/coordinators associated with one or more studies, and may provide coordinators view controls to filter the displayed data by various parameters (e.g., by study, country, site, coordinator, participant, etc.), to add user/coordinator, and to switch to study coordinator interface. Coordinators view controls may also be configured to switch to an individual coordinator interface receiving and displaying coordinator/user data 115, including coordinator assignments, coordinator sites, and coordinator participants. Individual coordinator interface may provide controls enabling participants' coordinator assignments to be modified or to be configured.

Institutions 95 receives from database 2 data about institutions (e.g., universities, hospitals, doctors' offices, medical facilities, and other entities) that may be associated with one or more studies or clinical sites, and may provide controls to filter the displayed institutions data by various parameters (e.g., study, site, location, etc.) and to add institutions.

Countries view 96 receives from database 2 data about various countries, for example, where participants or clinical sites associated with one or more studies are located and may also provide controls to filter the displayed countries by various parameters (e.g., study, site, etc.), and to switch to the country interface 97 (or just country 97). Country 97 (or country interface 97) comprises one or more panels and/or views, including, country studies (e.g., clinical studies 25 that involve the country), country sites (e.g., clinical sites serving the country), country users/coordinators (e.g., users who serve participants and or sites associated with the country), country participants (e.g., participants associated with the country), and county notes (e.g., notes related to the country).

Private cloud 6 may be any combination of networked computing resources (e.g., databases, servers, gateways, etc.) that are securely isolated from the internet (using, e.g., private networks, firewalls, secured gateways, etc.). In preferred embodiments, private cloud 6 is a virtual private cloud (VPC) (e.g., Amazon VPC, Google VPC, Rackspace VPC, Microsoft Azure) that uses shared computing infrastructure and resources, and isolates private cloud 6 resources, such as Database 2, servers 3, and others, using private subnets, virtual private networks (VPNs), encrypted channels, and/or other known methods. Embodiments may utilize a non-virtual private cloud 6 that uses dedicated computing infrastructure residing on or off premises, in a private data center, or with a managed private cloud provider (e.g., RackSpace, Cloudreach, etc.). Virtual private clouds can be implemented with hardware and software network security systems that are consistent with privacy law and regulations compliance (e.g., GDPR, HIPAA). Such features may include data encryption tools 11, monitoring tools 20, network gateways securing access to private cloud 6, such as internet gateway 7, API gateways 8, and others.

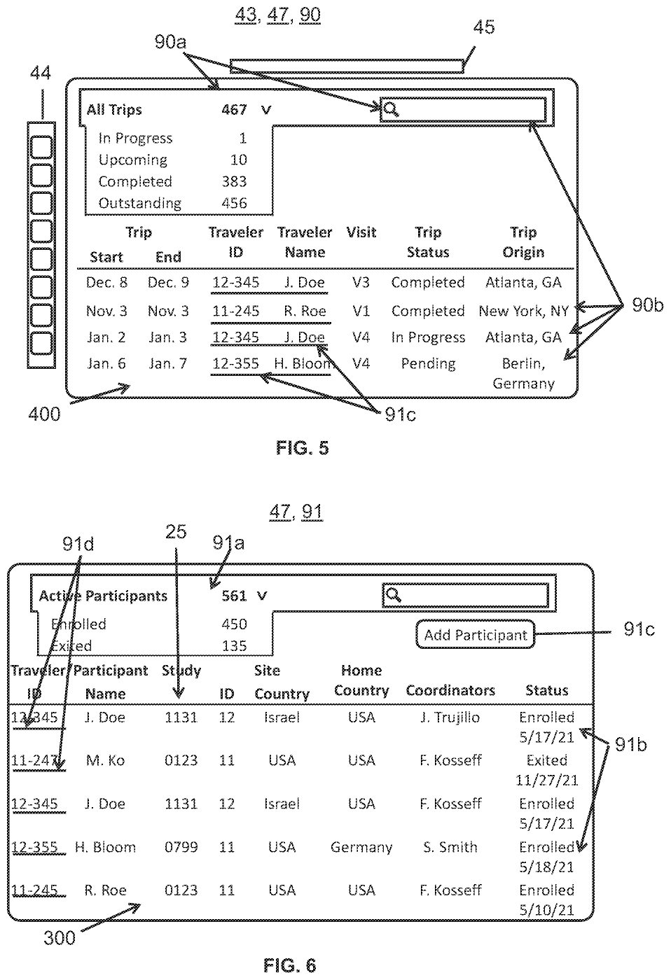

Encryption tools 11 may be configured to encrypt data 10 at rest, while stored in database 2, and to unencrypt data records of data 10 when those data records are accessed by GUI 43, or by another authorized computing resource. Encryption tools 11 may be configured to encrypt the entire database 2 (using, e.g., a transparent data encryption (TDE)), or specific records, cells, or data in Database 2. Encryption tools 11 may be configured to encrypt data being sent to Database 2 (e.g., client-side encryption), for example in steps 1101, 1102, 1104 of FIG. 3, and/or upon storing data in Database 2 (e.g., server-side encryption), for example in Step 1103. Encryption tools 11 may be software solutions separate from, or integrated with Database 2 and/or Private Cloud 6, and may be independently developed, or based third-party cryptographic services, solutions, or software development kits/toolkits (SDKs) (e.g., Azure Storage Service Encryption, Oracle Databases integrated TDE, AWS Encryption SDK, Google Cloud Encryption, IBM Guardium Data Encryption). Encryption tools 11 may utilize various encryption methods or algorithms, preferably cryptography encryption utilizing an encryption key 13 and a Key Management Service (KMS) system 14 that provides further security by separating the encryption key from encrypted Data 10. Encryption KMS systems can implement hardware or software encryption keys, separate the keys from data (e.g., only user external to the Database 2 has the key), handle the keys securely (e.g., allowing access to keys using a separate security policy), periodically generate new keys, and/or rotate keys. Various private cloud vendors provide encryption KMS systems, such as Google Cloud KMS, Amazon AWS KMS, Microsoft Azure Key Vault, Oracle Wallet, Oracle Key Vault, and others.

Internet gateway 7 may be utilized for enabling secure communications between the private cloud 6 and the internet, and enable secure transmission of personal information. In some embodiments private cloud 6 may comprise a public subnet (e.g., publicly accessible from the internet through internet gateway 7) and a private subnet isolated from internet gateway 7. In a preferred embodiment, private cloud 6, server 3, internet gateway 7, and workstation 40 communicate using https protocol utilizing transport layer security (TLS) encryption of all data transmissions between database 2 and workstation 40. Other methods for encrypting data 10 in transport may be used, either instead or together with TLS over HTTPS, for example, transmitting data 10 encrypted by encryption tools 11. For added privacy protection and data security workstation 40, internet gateway 7, server 3, and other devices within system 1, may be configured to prevent caching personal information to non-volatile storage (e.g., disk, ROM) and instead use only volatile computer memory (e.g., RAM) to transmit/display personal information.

Monitoring tools 20 may be utilized to monitor, log, and enable auditing of, access and communications between the internet, workstation 40, server 3, private cloud 6, and/or database 2. Monitoring tools 20 are known in the industry and may include cloud monitoring software or services (e.g., CloudWatch, MetricFire, Datadog, Dynatrace, Prometheus, Graphite, and others.), and logging tools (e.g., AWS VPC Flow Logs) that log and may provide audit trails for connections to private cloud 6 that attempt to access, process, transmit, and/or store data 10, including Personal Information. Monitoring tools 20 may also include private cloud monitoring services that monitor and log and identify all accounts (e.g., user or application) attempting to access private cloud 6 (including, e.g., source IP address, time of access attempts, etc.). Monitoring tools 20 may generate access logs for server 3, private cloud 6, and database 2, and store those logs for a period of time, preferably 60 months. Monitoring tools 20 preferably are within private cloud 6, but may also be external to private cloud 6, as illustrated in FIG. 1.

To implement granular access to private cloud 6, server 3, and database 2, system 1 may utilize an identity and security management (IAM) service or system 21. IAM 21 may identify, authenticate, and control access for individuals as well as hardware and applications that may need access to database 2, servers 3, or specific datasets or subsets, such as participant dataset 300 and allow users to only access data the users are authorized to access. For example, when a concierge-user is performing tasks through the GUI 43 (e.g., booking participant travel), an embodiment of the present invention may utilize IAM 21 to restrict GUI 43 from accessing and displaying Participant dataset 300 for participants who the user is not authorized to view. In a preferred embodiment, IAM System 21 is configured to restrict access to all Data 10 by user, and by user access level (e.g., user role) so that each user is allowed to access only the portion of Data 10 that the user is assigned or authorized to access. Access to server 3 also may be restricted by user, and user access level. User authentication may be handled internally, or by various existing platforms (e.g., Okta, OneLogin, AuthO, RSA SecurID, SecureAuth Oracle Access Management Suite). Embodiments of the present invention may also utilize multiple factor authentication in addition to username and password to increase data security.

Data 10 in database 2 may comprise one or more clinical study datasets 100 containing data subsets and information related to a plurality of clinical studies. The terms dataset and data subset generally are used interchangeably here, and should be understood broadly to include any set, collection, record or aggregation of data or information, in any form, created, received, or provided in relation to a clinical study 25 and can be of any size (including a single datum), and in any form, including, numbers, text, audio, video, images, documents, spreadsheets, and others.

Clinical study dataset 100 may comprise a plurality of data subsets (or sub datasets) about a clinical study 25, including protocol data 101, user/coordinator data 115, study visit schedule 130 dataset, clinical site dataset 200, participant dataset 300, and trip dataset 400. User/coordinator data 115 comprises information about users who are authorized to access the travel booking system, as well as users' assignments to one or more travelers. Protocol data 101, for example illustrated in FIG. 9, may comprise information that generally may be applicable to a study as a whole (e.g., the study protocol), and may include the study sponsor, study id, study name, study status, duration, start and end dates, drug or treatment information, study phase, medical or scientific area, appointment procedures 135 (e.g., drug or treatment administration, consultation, medical examination, MRI, CAT scan, PET scan, lab work, infusion, LP, surgery, PK sample collection, radiology, ultrasound), types of participants (e.g., gender, age, medical condition, environmental exposure, nationality, region, and others), and other relevant information.

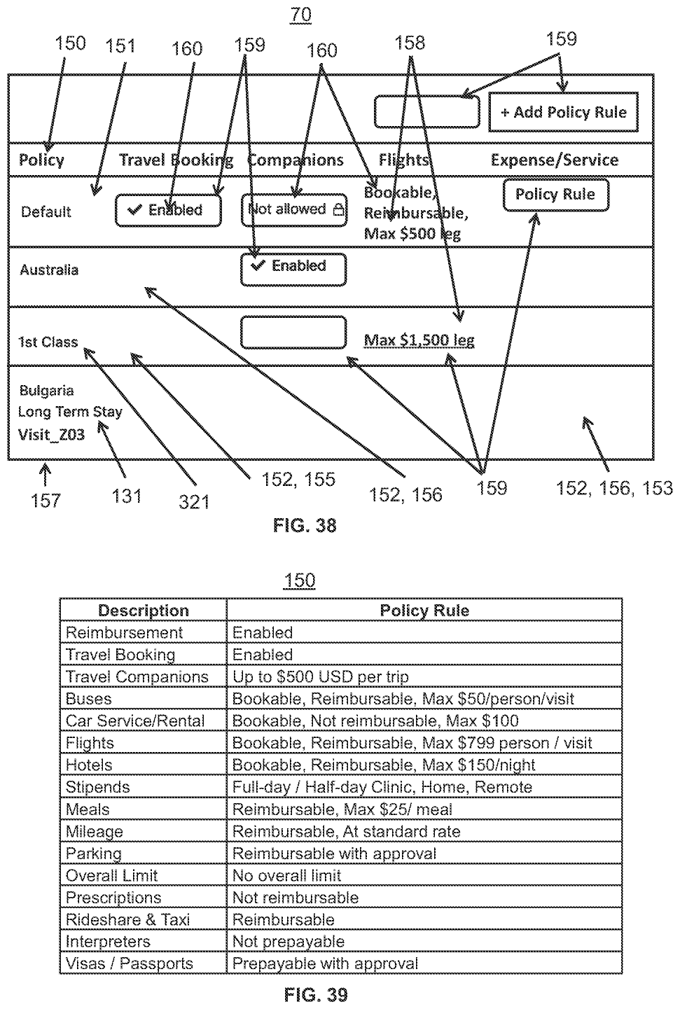

Study travel policy 150 (or study travel policy dataset 150) may comprise data about rules, options, restrictions, and policies applicable to travel by participants in a clinical study 25. For example, study travel policy 150 may include expense rules 158 comprising expense reimbursement information, such as, whether various travel related expenses (e.g., flight, cars, hotels, transportation, meals, tolls, parking, prescriptions, phone calls, and others) are reimbursable, maximum reimbursable cost, and whether approval is required. Study travel policy 150 may also include travel rules 160 that comprise indication whether certain travel services are bookable through system 1, and their maximum costs, such as air travel, car rental, car service, ferries, ambulances, rail, buses, hotels, long term lodging, and others. FIG. 39 illustrates an example of a study travel policy 150:

Study travel policy 150 may also comprise a main travel policy 151 and a specialized travel policy 152. Main travel policy 151 may apply by default to travel of clinical study participants 26, unless a specialized travel policy 152 applies to one or more aspects (i.e., travel policy conditions 157), of the travel.

A specialized travel policy 152 may establish specialized travel rules and restrictions 160 and/or specialized expense rules and restrictions 158 different from the main travel policy 151 main travel rules 160 and/or main expense rules 158, for example, different reimbursement amounts for some travel related services, different allowable cost for certain types of transportation or lodging (e.g., higher cost for flights, car services, higher or lower ranked hotels with different cost limits, permit long-term lodging), may allow booking of certain transportation that may not be allowed under main travel policy 152 (e.g., allow booking of car service, ambulance, or first class airfare that otherwise may not be allowed). For example, a specialized travel policy 152 may be a visit travel policy 153, site travel policy 154, custom travel policy 155, a country travel policy 156, and combinations thereof.

A specialized travel policy 152 may apply based on a travel policy condition 157. For example, a country travel policy 156 may apply based on country (e.g., origin, destination). A site travel policy 154 associated with a particular clinical site may apply for participant travel to and from the clinical site. A visit travel policy 153 may be based on (e.g., associated with) a visit policy label 131, and/or one or more visits 610 (a visit or visits having, e.g., a visit id 614, a visit type, a visit name 615, visit descriptions 622, and others). A visit policy label 131 may be associated with one or more visits or appointments (e.g., visit 1, visit “sg3,” “surgery” visits, visits 1-8, visits for baseline and screening during the same trip, visits during a clinical period 103, etc.).

A custom travel policy 155 may be based on a traveler policy label 321 associated with one or more participants 26. For example, a traveler policy label 321 may be a “1st class” label or a “long-term housing,” and a custom travel policy 155 associated traveler policy label 321 may allow booking of first-class air travel or long-term housing, even if main travel policy 151 does not allow them. A specialized travel policy 152 may also be based on various other factors (e.g., appointment location, institution, visit city or town, participant age, and others), as well as additional policy labels that may be associated with a participant, trip, site, country, institution, or other circumstances.

A specialized travel policy 152 may combine one or more of a visit travel policy 153, site travel policy 154, custom travel policy 155, and a country travel policy 156. For example, a specialized travel policy 152 combining a “long-term stay” visit policy label 131, a visit travel policy 153 for “surgery” visits, and a USA based country travel policy 156, will permit long-term hotel lodging in the USA for participants traveling for a “Surgery” visit associated with the “Long-Term Stay” label 131, even if the main travel policy 150 does not allow long term hotels.

Clinical site dataset 200 comprises information about a clinical site associated with a clinical study 25 where appointment procedures 135 are performed during clinical site visits to assess, evaluate, and/or examine participants 26. Alternatively, an appointment procedure 135 may be performed at a patient's home (or other non-site location) and the clinical site will perform the assessment upon receiving data from the appointment procedure 135. Certain appointment procedures may also be performed remotely, or virtually, using a mobile device, a portable analytical device, and similar equipment. Clinical site personnel may enroll participants in a study. A clinical site dataset 200 may comprise site appointment locations 201 information about one or more site locations, such as a site main location, and a site appointment location 201. A site appointment location 201 may comprise information about an appointment procedure 135, location address, (identified by, e.g., street, building, office or laboratory number), requirements or directions about participant 26 arrival (e.g., arrive at a different building, at a triage station, or wait for pick up at a different location). A site appointment location 201 may be the same or different from the clinical site's main location. A clinical site may perform all appointment procedures at a single site appointment location 201, or different appointment procedures may be performed at different site appointment locations 201. For example, a first site appointment location 201 may be associated with a first appointment procedure 135 (e.g., MRI, Cat Scans), and a second site appointment location 201 may be associated with a second appointment procedure 135 (e.g., medical observation, labs). Clinical site dataset 200 may comprise a site identifier, site name, and site contact information 202. Site contact information 202 contains information about who to contact (e.g., names, titles, departments, automated systems, etc.), whether the contact is to receive an itinerary 475 and how to provide it, and methods of contacting the clinical site (e.g., telephones, emails addresses, facsimiles, online access portals, API interfaces).

Clinical site dataset 200 may be provided by a clinical site and received by and stored into database 2 through the study sites interface 60 of GUI 43, as illustrated by numeral 1100a in the embodiment of FIG. 3. Clinical site dataset 200 may also be imported in the form of a data file (e.g., CSV, etc.), or it may be transmitted electronically over a network using secure transfer protocols (e.g., FTPS, UDP, etc.) exemplified by numeral 1101.

Participant dataset 300, comprises data about a participant 26 in a clinical study 25, including participant study information 301, participant personal data 307, traveler profile 309 data, and other data.

Participant dataset 300 may be received in database 2 using various techniques and methods, an example of which is illustrated in FIG. 3. In step 1100 clinical sites may enroll patients 26 into a clinical study 25. Embodiments of system 1 may receive participant dataset 300 from the clinical sites either directly (if transmitted electronically) as indicated in step 1102, or via input device 41, if participant data set is provided in hardcopy or as electronic file as illustrated in steps 1100a and 1105. In step 1105, a user may input participant dataset 300 through participant interface 52 of GUI 43 utilizing input device 41. In step 1104 GUI 43 may transmit participant dataset 300 to database 2 where it is securely stored in step 1103. Participant dataset 300 may be transmitted electronically (using, e.g., API, FTP, UDP, secure file transfer protocols, etc.) from the clinical site or another entity as illustrated by step 1102, and then stored in database 2 in step 1103. A user may also add additional participant data 300 through GUI 43, in a participant “onboarding” process by obtaining and inputting additional participant information to supplement, customize, or individualize various portions of participant dataset 300. The onboarding process may include phone calls, electronic assistance request forms, and other methods to obtain information from participants or other relevant sources. A request to access a secure electronic assistance request form may be transmitted to clinical site personnel, participants, caretakers, and other parties with relevant information and will allow them to securely input the information in participant dataset 300.

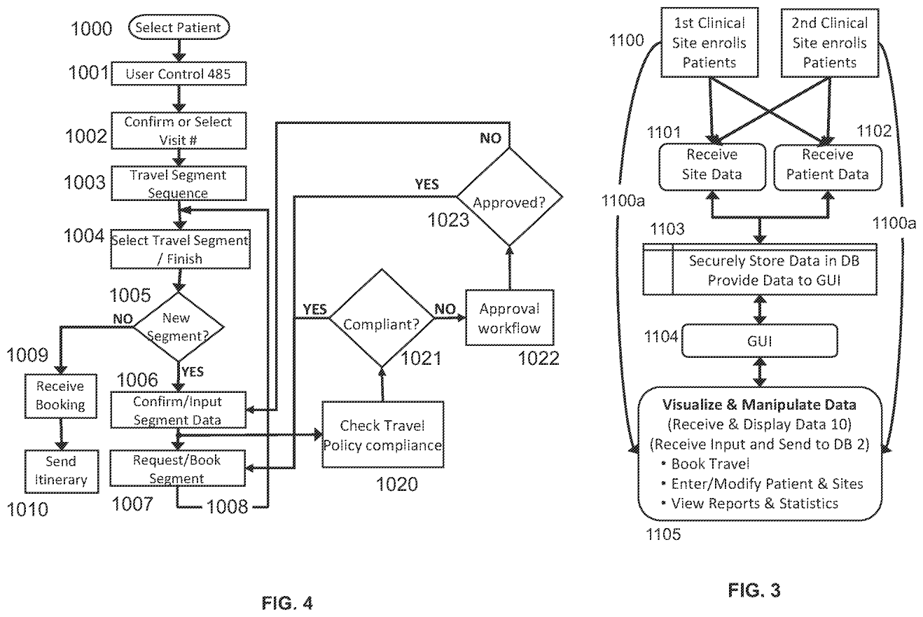

For the privacy and security of the participant dataset 300, it may be stored encrypted in database 2 identifiable by a unique identifier 12. Unique identifiers 12 may be associated with the separate data records of participant dataset 300 and utilized to pseudonymize participant dataset 300 and enhance security of personal information. For example, participant dataset 300, or any portion of it may only be accessed by utilizing one or more unique identifiers 12, instead of actual information about participant 26. FIG. 2 illustrates an example in which GUI 43 requests (arrow labeled as “request”) from database 2 travel profile dataset 309 for a participant 26 with a unique identifier “1234aabbcc.” GUI 43 will receive (arrow labeled as “receive”) from database 2 a record containing travel profile 309 corresponding to the requested unique identifier 12.

Participant study information 301 comprises participant study id 302 that references a participant 26 in a clinical study 25 without revealing participant name or other personal information. Participant study id 302 preferably is a unique code associated with a participant, and may consist of a clinical site id of participant assigned site 303, and an additional alphanumeric sequence. Participant id 302 may be displayed in GUI 43 or provided in various documentation and may be utilized by parties associated with a study to view study information without revealing a participant's name or other personal information. If a participant invokes a right to forget personal information (e.g., privacy laws), participant id 302 may be used to identify and purge the appropriate personal information. Participant study information 301 may also comprise an enrollment date, participant consent record 330, participant assigned site 303, participant visit data 304, participant country 305, and a traveler policy label 321. Participant Id 301 may be associated with a unique identifier 12 corresponding to participant 26, with a participant study id 302, or with a study enrollment id 12a. As an example, FIG. 37 illustrates GUI 43 receiving from database 2 a study travel policy 150 associated with a participant 26 whose participant id 301 is 123-4576, unique identifier 12 is “123aabcc”, and enrollment id 12a is “93ae34c.”.

Participant consent record 330 may contain information about a participant's informed consent related to access, use and/or processing of personal information related to a clinical study 25, for example for travel booking, medical assessments, and other purposes. Participant consent record 330 may comprise data indicators (e.g., in the form of binary flags, such as “0” or “1”, “true” or “false”) that a participant has provided informed data consent in the form of a consent provided indicator 3301, or that a participant has withheld, or not yet provided, informed data consent in the form of consent not provided indicator 3300. Consent provided indicator 3301 and consent not provided indicator 3300 may be distinct indicators or may be a single binary (on/off, true/false) indicator, so that on or true indicates consent provided, and off/false indicates consent not provided. Participant consent record 330 is required for all participants (including their companions or caretakers whose personal information may be needed). Participants must sign a consent to process personal data (including health data), which may vary depending on the applicable jurisdiction (e.g., US, GDPR/non-GDPR countries, Australia, Russia, Turkey, Canada, others).

Participant consent record 330, and consent provided indicator 3301 or consent not provided indicator 3300 may be more granular and further comprise indicators of whether participant has provided consent regarding some “special” types of personal information data but not others, such as personally identifiable information (PII), personal data (e.g., PD under GDPR), protected health information (e.g., PHI under HIPAA, “Data concerning health” under the GDPR), and whether an electronic copy of an appropriate signed consent form is included in record 330. In a preferred embodiment an appropriate participant consent record 330, evidencing participant's 26 consent for both personal information and for health records, as well as images of the executed consent forms are required for a consent provided indicator 3301 to be present. Embodiments may require a consent provided indicator 3301 to enable the creation and storage of participant 26 data, including participant personal data 307, traveler profile 309, and others, and may be configured to prevent the creation or entry of a participant dataset 300, or to initiate travel booking, for any participant or companion without a consent provided indicator 3301. Embodiments of the present invention may generate an error message, create notifications, and/or prevent storing of any portion of a participant dataset 300 that does not contain data in the participant consent record 330.

Consent provided indicator 3301 and/or consent not provided indicator 3300 may be set automatically by the system based on the presence or absence of signed consent forms and other factors or may be manually set by a user. In an embodiment, a participant (or companion) may view and e-sign disclosure and consent forms for the processing of their personal information where such forms may be provided electronically in a similar manner as the assistance request form described above.

Participant assigned site 303 comprises information about a clinical site associated with a participant in a clinical study 25. A participant may have more than one participant assigned site 303 so that the participant may visit different sites and different times during a study.

Participant visit data 304 represent the visit schedule associated with an individual participant (which track, which visits, etc.) and where (progress) of the participant in the visit schedule, all that based on the study visit schedule 130 applicable to the entire study. Participant visit data 304 comprise the visit schedule applicable to a participant based on study visit schedule 130 data, including information about at least one appointment procedure 135 and its associated appointment schedule 136 (e.g., start date/time, end date/time, duration 621, and other attributes). Participant visit data 304 may also comprise information about visits that have already occurred, and a current visit 306 (e.g., visit name 615, and/or number of the next visit that needs to be scheduled). Participant visit data 304 may also associate a participant's trip with a specialized travel policy 152 based on a visit policy label 131 in study visit schedule 130. For example, if a participant is traveling for a visit (e.g., an appointment) with a visit id 614 or visit name 615 associated with a visit policy label 131 in study visit schedule 130, portions of participant's trip (e.g., trip segments) may be subject to a specialized travel policy 152 associated with the visit policy label 131 if applicable. Participant visit data 304 may also comprise a traveler policy label 321 associated with a custom travel policy 155.

Participant personal data 307 comprise identification and other information about a participant, such as name, sex, gender, birthdate, age, personal details, languages, government issued ids, known traveler numbers, and other information. Participant personal data 307 may include participant documents 310 in the form of uploaded electronic files (e.g., pdfs, images, QR Codes) containing information about and copies of passports, government issued ids, travel documents, medical cards, health forms, and others. To maintain security and data privacy, when electronic files with personal information are uploaded, preferably they are stored in heavily restricted logical storage locations (e.g., AWS S3 buckets, Azure Blob Storage) encrypted using encryption keys, and identifiable only via one or more pseudonyms, which may include a participant id, a unique identifier 12, and similar data security constructs. Preferably, all data 10, including files (e.g., consent forms, passports, expense receipts) are stored into database 2 and associated with an individual unique identifier 12, for example as illustrated in FIG. 23. In addition, each record of data 10 may also be associated with a unique identifier 12 of a participant 26, facilitating scrubbing of personal information.

Participant personal data 307 may further comprise traveler contact information 308, such as an address, phone, email, primary contact (e.g., the participant, caretaker), and contact instructions (contact by, e.g., phone, email, sms).

Traveler profile 309 may comprise data about traveler origin location 314, traveler identification 311, traveler health restrictions 312, and traveler preferences 313. Participant Traveler profile 309 may also comprise a traveler policy label 321 associated with a custom travel policy 155.

Traveler origin location 314 may identify a street address for a participant (e.g., a home address, family home, hospital, facility, government building) as the preferred origin location for a participant's travel for a clinical study 25. Traveler origin location 314 also may comprise different traveler origin locations 314 depending on the mode of transportation, for example, a street address for car service pick-up or drop-off, or the traveler origin location 314 may be a preferred departure airport for flights according to a traveler flight preferences 341, a preferred bus terminal, train station, or port according to bus preferences 342, rail preferences 343, or ferry preferences 344, respectively.

Traveler personal identification 311 may comprise a name, contact information 308, participant documents 310, known traveler number, and other information needed to book a flight, a hotel, reserve a car, or travel internationally.

Traveler health restrictions 312 comprises information about needed travel accommodations based on the traveler's health, medical conditions, and personal characteristics. For example, traveler health restrictions 312 may comprise information about a traveler's ability & assistance requirements, if a traveler has a vision or hearing impairment, does not speak the local language, requires a wheelchair or another assistive device, has cognitive or developmental disabilities, requires animal assistance, and others. Traveler health restrictions 312 may also comprise notes to assist a concierge-user while booking travel.

Traveler preferences 313 may comprise information about travel and lodging preferences or requirements, including flight preferences 341, hotel preferences, car service preferences 345, car rental preferences 346, bus preferences 342, rail preferences 343, ferry preferences 344, hotel preferences 347, long-term housing preferences 348, and other travel preferences 313. Traveler preferences 313 may also comprise information about ambulance use and other conditions, such as supplemental oxygen, feeding tube, a colostomy bag, and others.

Flight preferences 341 may comprise information about preferred departure airports and airlines, seats, governmental security programs (e.g., TSA, known traveler numbers), frequent flyer programs, and other air travel related information. Flight preferences 341 may also comprise information about allergies, need for an additional seat or for wheelchair assistance on and off the plane, travel with an oxygen tank, and other Americans with Disabilities Act (ADA) requirements or restrictions, and other information about travel circumstances.

For example, bus preferences 342, rail preferences 343, and ferry preferences 344, may comprise information about a preferred departure port or station, transportation provider, and type/class of seating; boarding assistance needs; rewards programs, and other personal and health information. Car service preferences 345 and car rental preferences 346 may comprise information about a preferred service or rental company, vehicle class, pick up or drop off locations; wheelchair or other assistive needs, child safety seat requirement; and other information about traveler's abilities and preferences. Lodging preferences, such as hotel preferences 347 or long-term housing preferences 348 may comprise information such as a preferred hotel, room floor, type and size, wheelchair accessibility, allergen free, rewards programs, parking, need for service animals, and any other ADA requirements or restrictions.

Companion dataset 325 may comprise information about travel companions, (e.g., caregivers, guardians) of a participant. A travel companion may be a participant associated with a participant dataset 300, or a non-participant associated with participant companion dataset 300, comprising information necessary for travel.

Trip dataset 400 about a traveler 26 trip may comprise a trip origin 401, trip destination 402, trip schedule 403, trip segment data 501, itinerary 475, and booked travel data 450. Booked travel data 450 may comprise segment booking information 508 for booked, reserved, or confirmed trip segments, and also may comprise traveler identification 311. Booked travel data 450 may also comprise price quote information for trip segments 501. Booked travel data 450 may vary based on trip segments. For example, for a flight segment booked travel data 450 may comprise a ticket and flight number, record locator, cost, airports, airlines, seating, schedule, customs and immigration information, luggage limitations, security warnings and requirements, and other air travel data. Hotel segment booked travel data 450 may comprise a hotel name and address, reservation number, cost, room type and amenities, hotel rules, policies, and other hotel information. An appointment segment booked travel data 450 may comprise visit (or appointment) confirmation, appointment schedule 136, appointment procedure 135, instructions such as direction to appointment location, access codes, preparation, and other information.

FIGS. 27, 32-35 illustrate an exemplary itinerary 475 comprising one or more of an itinerary message 478, booked travel data 450, trip segment data 501 (including segment booking information 508), traveler identification 311, traveler contact information 308, and other data from trip dataset 400 or participant dataset 300.

Trip origin 401 may comprise information about a trip's initial departure location, and may be based on (or determined according to) a traveler origin location 314, study travel policies 150, and a beginning trip segment data 501 (e.g., a trip's first trip segment). For example, if the beginning trip segment is a car service segment 514, trip origin 401 may be a street address provided in traveler origin location 31. In another example, if a trip requires air travel and study travel policy 150 does not permit a car service, the beginning trip segment may be a flight segment 512 and trip origin 401 may be a preferred departure airport in traveler origin location 314. Trip destination 402 may comprise information preferably based on a site appointment location 201 including address, directions, and other requirements. When a trip dataset 400 comprises one trip segment, such as an appointment segment 511, travel origin 401 and travel destination 402 may be the same (e.g., correspond to a site appointment location 201).

Trip schedule 403 may comprise data about one or more of a departure time, a duration, and a return time (e.g., time of departure for the return trip). Trip schedule 403 preferably is based on one or more of appointment schedule 136 and trip segments 501. For example, trip schedule 403 departure time may be determined based on appointment schedule 136 and outbound trip segment schedules 505 so that a participant arrives at trip destination 402 before or at the appointment schedule 136 appointment start date and time, or in some embodiments, before an arrive by date and time 137. In another example, trip schedule 403 return time may be the departure time for a return trip and may be determined based on appointment schedule 136 so that a participant departs appointment location 201 after the appointment end date and time, or after the depart date and time 138.

Trip segment data 501 may be data about an appointment segment 511, a flight segment 512, a bus segment 513, a car service segment 514, a car rental segment 515, a rail segment 516, a ferry segment 517, an ambulance segment 518, and a lodging segment 519. Embodiments may comprise a trip dataset 400 having a first trip segment data 501, for example, an appointment segment 511, and a second trip segment data 501, for example a flight segment 512.

Trip segment data 501 (or trip segment 501) may comprise information about a segment start location 503, segment end location 504, segment schedule 505, segment status 506 (e.g., booked, booking requested, pending, confirmed, reserved, etc.), segment instructions 507, segment booking information 508, and information. Segment instructions 507 may comprise messages (e.g., allergies, health and medical needs, preferences) to the trip segment provider (e.g., airline, car company, hotel) based on the traveler profile 309 (e.g., traveler health restrictions 312, traveler preferences 313), as well as information/directions for a traveler (from, e.g., clinical site dataset 200).

Trip segment data 501 may be determined (by system 1 or a user) based on data from traveler profile 309, study travel policies 150, visit schedule 130, clinical site dataset 200, and a previous or subsequent trip segment 501. For example, a beginning trip segment start location 503 may be the trip origin 401, or the segment start location 503 for the first trip segment 501 of a return leg of a trip may be the trip destination 402 (e.g., appointment site location 201). In another example, a first segment end location 504 may be based upon (e.g., same as, nearby) a second segment start location 503 of a second (e.g., subsequent to the first) trip segment 501. Similarly, a second trip segment start location 503 may be based on, or be the same as, the first segment end location 504. Trip segment data 501 may comprise maps or directions (e.g., from google maps, street view, hotels or clinical site photos) related to trip segment start location 503, trip segment end location 503, and data from previous or subsequent trip segment data 501.

Trip segment schedule 505 (or just segment schedule 505) may comprise a segment start time (e.g., departure time, check-in time), segment duration, and/or a segment end time (e.g., arrival time, check out time). For example, a first segment schedule 505 (e.g., start time, end time) may be based upon a second (e.g., preceding or following the first) segment schedule 505 (e.g., end time, start time). Beginning trip segment schedule 505 and the initial trip segment 501 of a return leg of a trip may be based on the trip schedule 403 departure time and trip schedule 403 return time, respectively.

Segment booking information 508 comprises information necessary to book or reserve a trip segment determined based on traveler profile 309 (e.g., traveler health restrictions 312, traveler preferences 313), and applicable study travel policy 150 (e.g., maximum cost, permitted trip segment or services classes, etc.). Segment booking information 508 may also comprise information for a traveler based upon booked travel data 450 (e.g., flight, bus, or train numbers; reservation number; rental car model, color or license plate; ambulance provider; seat numbers; hotel name), clinical site dataset 200 (e.g., site contact, access instructions), and other helpful information.

Appointment segment 502, comprises data about a patient visit to a clinical site for clinical study assessment, and may be determined based on study visit schedule 130 and/or clinical site dataset 200, including for example appointment schedule 136, appointment procedure 135, and appointment location 201.

Flight segment data 512 may be a one-way flight segment 512 having an outbound flight 512a, or a round trip flight segment 512 having an outbound flight 512a and a return flight 512b. Flight segment booking information 508 may comprise information necessary to book all flights in a flight segment 512 determined based on data from one or more of traveler profile 309, including for example, flight preferences 341, study travel policy 150, and traveler identification 311. Segment booking information 508 for a flight segment 512 may also comprise data from booked travel data 450, including airports, terminals, flight numbers/carriers, confirmation/ticket numbers, baggage information, and other flight information.

Flight segment start location 503 may be determined based upon traveler profile 309 (e.g., preferred airport, distance from traveler home address), or segment end location 504 of a preceding trip segment 501 (e.g., a rail segment 516, another flight segment 512, appointment segment 511). Flight segment 512 segment end location 504 may be based upon traveler profile 309 and/or a subsequent travel segment (e.g., appointment location 201, lodging segment 513 location, bus segment start location). A round trip flight segment start location 503 may be the same as the round trip flight segment end location 504 (e.g., same arrival and departure airport). In such an example, outbound flight 512a end location 504a may be the same as return flight 512b start location 503b. Flight segment schedule 505 may comprise flight departure and arrival times, as well as flight durations, for all connecting outbound and/or return flights 512a, 512b, of a flight segment 512.

Lodging segment 513 may be a hotel segment 513 or long-term lodging segment 513. Lodging segment booking information 508 may comprise room type, floor, size, accessibility, preferred or booked hotel or lodging company, lodging confirmation and/or reservation numbers, and other information based on traveler profile 309 (e.g., hotel preferences 347, long-term housing preferences 348), study travel policy 150, and booked travel data 450. Lodging segment start location 503 (preferably the same as end location 504) comprising a lodging address, may be based upon traveler profile 309 (e.g., preferred hotel), distance from a preceding or subsequent trip segment (e.g., appointment location 201, arrival or departure airport, etc.). Lodging segment schedule 505 (date/time to, e.g., check-in and check-out times) may be determined based on appointment schedule 136, traveler profile 309, travel policy 150, and preceding or subsequent trip segments (e.g., arrival and departure dates/times).

Surface transportation trip segments 501 such as rail segment 343, bus segment 342, and ferry segment 344, may comprise data about a one-way or round trip surface transportation, including, surface transportation segment booking information 508 (e.g., train, bus, ferry, or route numbers, transportation company, confirmation/ticket numbers, baggage information) and other information based on one or more of traveler profile 309 (e.g., rail preferences 343, bus preferences 342, ferry preferences 344), study travel policy 150, and booked travel data 450. Surface transportation segment start location 503 may be determined based on traveler profile 309 (e.g., preferred station or port, distance from traveler home address), previous segment end location 504 and other information. Surface transportation segment end location 504 may be based on traveler profile 309 and clinical site dataset 200 (e.g., appointment location 201, arrival instructions). Surface transportation segment end location 504 may be based on a subsequent trip segment start location 503. Segment schedule 505 of a surface transportation trip segment 501 may comprise departure and arrival times and segment durations, for all connecting outbound and/or return train, bus, or ferry routes within a surface transportation trip segment 501.

Car service segment 514 and car rental segment 515 (also referred together as “car segment 514, 515”) booking information 508 (e.g., vehicle number/license plate, car company, confirmation numbers, luggage information) may be based on one or more of traveler profile 309 (e.g., car service preferences 345, car rental preferences 346), study travel policy 150, and booked travel data 450. Car segment start location 503 (e.g., pick-up location) may be determined based upon traveler profile 309 (e.g., traveler origin location 314), or a previous trip segment end location 504 (e.g., airport, appointment location). Car segment end location 504 (e.g., a drop off location) may be determined based on a traveler's home address, or on a subsequent trip segment start location 504 (e.g., airport, appointment location). Segment schedule 505 of a car service segment 514 may comprise pick-up and drop-off times, as well as trip duration.

Travel planning interface 500 of GUI 43 provides an easy and efficient process for a travel coordinator (e.g., user, concierge-user) to plan and book a trip for a traveler. GUI 43 may be configured to switch to the travel planning interface 500 by utilizing controls as described above.

Travel booking module 575, illustrated in FIG. 1, may be any type of system, software, device, entity, or combinations thereof, configured to i) receive booking request data 451 about a trip or trip segments from travel planning interface 500, ii) book, or obtain a quote for, the trip or trip segment based on the booking request data 451; and iii) transmit or provide to travel planning interface 500 booked travel data 450 about the trip or trip segment. Booking request data 451 comprises one or more of trip segment data 501, including segment booking information 508 (for each trip segment 501, e.g., desired start and end locations 503, 504, schedules 505, traveler restrictions and preferences) determined during travel booking using travel booking interface 500, and traveler identification 311 needed to obtain a booking (e.g., for flights, international travel, hotels, etc.).

Travel booking module 575 may comprise a browser interface that may be separate or distinct from GUI 43 and/or travel planning interface 500, may be networked with GUI 43, or may be incorporated (e.g., as a separate panel, interface, frame) within GUI 43 or within travel planning interface 500. Such travel booking module 575 may be configured to enable a user to access travel websites, and search for, select, and/or book, appropriate trip segments (e.g., flight, train, bus, hotel, car rental, car service) based on booking request data 451. Travel websites may include website of travel providers, such as airlines, railroads, bus companies, hotels, car rentals or service companies, ambulance operators, and others, or may include travel booking websites (e.g., booking.com, orbitz.com, travelocity.com, expedia.com, hotels.com, carrentals.com). Travel booking module 575 may also comprise, or include the services of, virtual or conventional travel booking agents (e.g., independent or affiliated with a travel company), including general travel agencies, agencies specializing in a particular type of transport (e.g., car service, long-term housing, ambulance, medical transportation, etc.), and other types of booking agents. Travel booking module 575 may also comprise an interface to a clinical site (e.g., a clinical site scheduling personnel or system) to book (e.g., confirm) an appointment segment 511.

Travel booking module 575 preferably will book, or obtain quotes (automatically and/or with human involvement), for a trip or trip segment based on received booking request data 451 (e.g., a flight between requested airports on a requested date, requested hotel reservation for a duration appropriate for traveler restrictions), and transmit to travel planning interface 500 booked travel data 450 including booking or quote information about the trip or trip segment. Segment booking information 508 may be updated with booked travel data 450 received from travel booking module 575.

Booking request data 451 may be transmitted by travel planning interface 500 electronically over a network (using, e.g., API, URL incorporating trip segment information, data files with trip information, email), by telephone, or from input device 41 (e.g., when travel module 575 is a travel booking website). Embodiments of travel planning interface 500 may also transmit booking request data 451 by storing it in database 2, and causing system 1 (e.g., servers 3) to access booking request data 451 in database 2 and send it to travel booking module 575, for example, using the above-described methods. Travel planning interface 500 may receive booked travel data 450 similarly over a network, by email, in the form of printouts, from database 2 (e.g., if servers 3 received and stored booked travel data 450).

GUI 43 provides an efficient access to travel planning interface 500 from various views and panels by utilizing, for example, controls 483, 485 associated with a participant 26 (from, e.g., individual trip module 59, participant interface 82, etc.). Travel planning interface 500 comprises trip segment controls and trip segment panels 520 that can be utilized to correct, enter, customize, and confirm information in trip segment data 501 for each trip segment and for each participant, for example by personalizing trip segment data 501 for each participant, adding notes, detailed directions and instructions, and personalized requests.

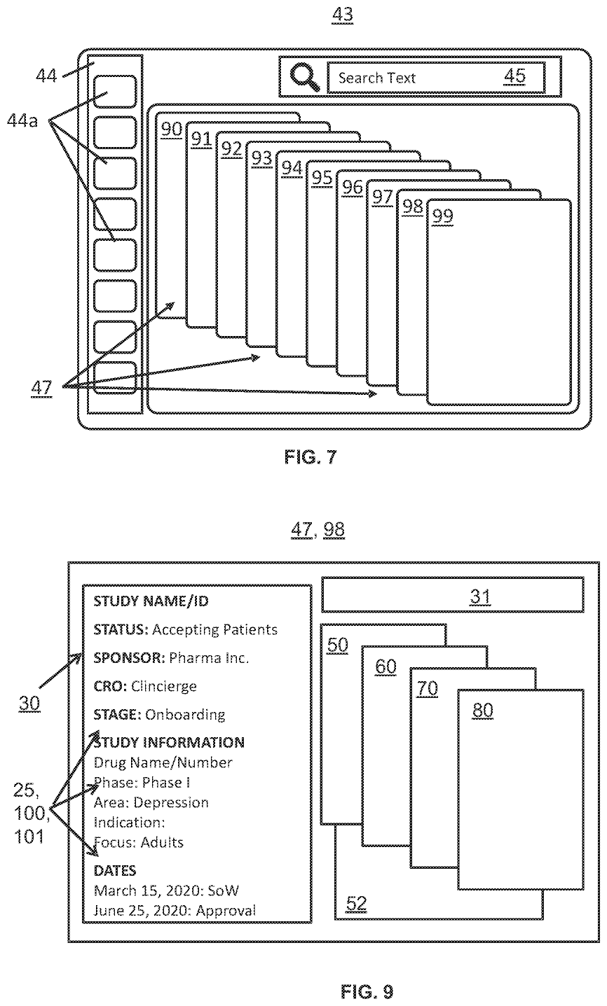

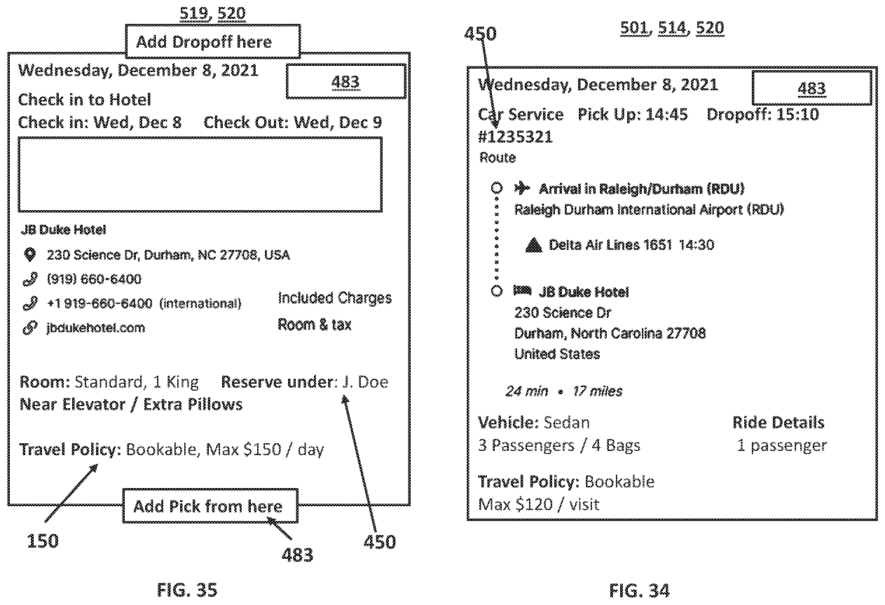

An embodiment of the invention may be utilized and function as illustrated in the flowchart in FIG. 4. In step 1000 a user may utilize control 485, 482, to select a participant 26 as indicated in step 1001, and to initiate trip planning interface 500. Trip planning interface 500 receives from database 2 a trip dataset 400 associated with patient 26 and participant visit data 304. In step 1002, current visit 306 from patient visit data 304 may be displayed for confirmation (also allowing a selection of a different current visit 306 prior to confirmation). Upon confirming a current visit 306 the travel interface 500 advances to step 1003 and the GUI switches to a sequence of segments panels 520 providing controls 483 allowing the addition of a trip segment 501. In step 1004 another trip segment 501 may be selected using control 483 (e.g., selecting a flight, car service, hotel, or other segments from a menu) as illustrated in FIG. 36, or control 483 may be used to indicate that no more segments will be added (e.g., clicking “finish”). Depending on study travel policy 150 applicable to the trip or patient 26, some types of trip segments may or may not be available. For example, if the main travel policy 151 does not allow bookable car service, car service segments may not be able to be booked (or may require approval), unless a specialized travel policy 152 that allows car service applies (e.g., country policy based on origin or destination, visit policy, etc.). In such an example, if patient 26 or current visit 306 are subject to a specialized travel policy 152 (based on, e.g., patient country, visit 306 type, etc.) car service segment may be available for selection in step 1004.

If a new segment is selected, indicated with “yes” in step 1005, travel planning interface 500 advances to step 1006 displaying the appropriate trip segment panel 520 (e.g., flight segment panel, hotel segment panel, appointment segment panel, etc.) with appropriate trip segment data 501. Trip segment data 501, and preferably segment booking information 508, may be determined as described above based on traveler profile 309, study travel policies 150, current visit 306 (e.g., appointment procedure, schedule), clinical site dataset 200 (e.g., appointment location associated with the appointment procedure), and trip segments data 501 for previous or subsequent trip segments that have already been generated for the trip using travel planning interface. Trip segment data 501 may also be determined based on study travel policies 150 applicable to patient 26 and current visit 306. For example, if patient 26 is associated with a traveler label 321 (e.g., first class allowed) a flight trip segment data may include information about first class tickets; or if current visit 306 is associated with a visit travel policy (based on, e.g., a visit type or id, or visit label 131) that allows long-term lodging, lodging segment data may include information about long term lodging.

Trip segment panel provides controls that may be utilized to modify, select, input, and confirm trip segment data 501. In step 1007, the travel planning interface 500 enables a user to “request booking” by transmitting to travel booking module 575 booking request data 451, comprising trip segment data 501, preferably including segment booking data 508, determined in step 1006 for a trip segment selected in step 1004.

After transmitting booking request data 451 to travel booking system 575 the travel planning interface 500 returns to step 1004, as indicated by arrow 1008, allowing the selection of another trip segment 501 or to finish planning the trip. If another trip segment is selected, travel booking interface 500 repeats steps 1005 1007. If no more trip segments are needed, indicated by a “no” in step 1005, the travel interface 501 advances to step 1009 to receive the booked travel data 450 from travel booking module 575. Embodiments may be configured to transmit booking request data 451 for each trip segment 501 in step 1007. Other embodiments may be configured to include an aggregation step (not shown) where booking request data 451 are aggregated for all trip segments 501, and transmitted as booking request data 451 for an entire trip.

Embodiments may be configured with an additional check for travel policy compliance, indicated by steps 1020-1023, preferably when a system 1 is configured to permit non-compliant travel upon approval. After a confirmation of trip segment data 501 in step 1006, the in step 1020 a computing algorithm compares trip segment data 501, and preferably segment booking information 508, to the applicable study travel policy 150 (e.g., cost, whether certain services are bookable or not, and other) including main travel policy 151, and any specialized travel policy 152 applicable to the trip being booked (e.g., based on individual traveler policy labels 321, countries, current visit 306, visit policy label 321, and others). If trip segment data complies with all applicable study travel policies 150, indicated as “YES” in step 1021, the travel interface 500 continues to step 1007. If the algorithm results in non-compliance (e.g., “NO” in step 1021), travel planning interface 500 invokes an approval workflow 1022 (which may involve escalation to management, or other steps). If the approval workflow 1022 results in approval in step 1023 (“YES”), the travel interface 500 advances to step 1007. If workflow 1022 results in a rejection in step 1023 (“NO”), travel interface 500 returns to step 1006 allowing the user to modify or input new trip segment data.

In step 1009, trip planning interface 500 may generate an itinerary 475 as described above (and including received booked travel data 450) and may enable GUI 43 to switch to individual trip 59 module for further actions, such as sending notifications, entering additional trip information, or modifying travel segments.

While the invention has been described with reference to exemplary embodiments, it will be understood by those skilled in the art that various changes, omissions, and/or additions may be made and equivalents may be substituted for elements thereof without departing from the spirit and scope of the invention. In addition, many modifications may be made to adapt a particular situation or material to the teachings of the invention without departing from the scope thereof. Therefore, it is intended that the invention is not limited to the embodiments disclosed as the best mode contemplated for carrying out this invention, but that the invention will include all embodiments falling within the scope of the appended claims. Moreover, unless specifically stated, any use of the terms first, second, etc., do not denote any order or importance, but rather the terms first, second, etc., are used to distinguish one element from another.

We claim:

1. A system for visualization and management of a travel policy for a clinical study, the system comprising:
a study dataset comprising a first travel policy condition;
a study travel policy dataset comprising a main travel policy and a specialized travel policy; and,
a GUI comprising a travel policies interface and a travel policy control;
wherein the first travel policy condition is selected from the group consisting of a country, a clinical site, a traveler policy label, a visit attribute, a visit policy label, and combinations thereof;
wherein the specialized travel policy is programmatically associated with the first travel policy condition;
wherein the specialized travel policy is selected from a country policy, a site policy, a first visit policy, a second visit policy, a custom policy, and combinations thereof;
wherein the GUI receives from the database the travel policy condition and study travel policy dataset;
wherein the travel policies interface displays the travel policy condition, the main travel policy, and the specialized travel policy;
wherein the travel policies interface is configured to visually associate the first travel policy condition with the specialized travel policy;
wherein the GUI is configured to enable using the travel policy control to modify the specialized travel policy; and,
wherein the GUI, responsive to using the travel policy control to modify the specialized travel policy, is configured to (a) receive a modified specialized travel policy, (b) store the modified specialized travel policy in the database replacing the specialized travel policy, (c) cause the database to programmatically associate the modified specialized travel policy with the first travel policy condition, and (d) update the travel policies interface to visually associate the travel policy condition with the modified specialized travel policy.

2. The system of claim 1, wherein the GUI is configured to enable using the travel policy control to add a second travel policy condition;
wherein the GUI, responsive to using the travel policy control to add the second travel policy condition, is configured to (a) receive the second travel policy condition, (b) store the second travel policy condition in the database, and (C) update the travel policies interface to display the second travel policy condition;
wherein the GUI is configured to enable using the travel policy control to add a new specialized travel policy; and,
wherein the GUI responsive to using the travel policy control to add the new specialized travel policy, is configured to (a) receive the new specialized travel policy, (b) receive information identifying the first travel policy condition or the second travel policy condition, (c) store the new specialized travel policy in the database, (d) cause the database to programmatically associate the new specialized travel policy with either the travel policy condition first or the second travel policy condition according to the information identifying the first travel policy condition or the second travel policy condition, and (e) update the travel policies interface to display the specialized travel policy in visual association With either the first travel policy condition or the second travel policy condition according to the information identifying the first travel policy condition or the second travel policy condition.

3. The system of claim 2, wherein the main travel policy comprises a main travel rule and a main expense rule,
wherein the specialized travel policy, the modified specialized travel policy, and the new specialized travel policy, each comprise a specialized travel rule, a modified specialized travel rule, and a new specialized travel rule, respectively;
wherein the travel policies interface displays the first travel policy condition in visual association with the specialized travel policy; and,
wherein the travel policies interface displays the second travel policy condition in visual association with the new specialized travel policy.

4. The system of claim 3, wherein the travel policies interface displays the first travel policy condition, the second travel policy condition, the specialized travel policy, and the new specialized travel policy as a table;
wherein the first travel policy condition is displayed visually aligned with the specialized travel rule and the specialized expense rule; and,
wherein the second travel policy condition is displayed visually aligned with the new specialized travel rule and the new specialized expense rule.

5. The system of claim 3, wherein a clinical dataset comprises a first participant dataset;
wherein the first participant dataset comprises the first travel policy condition; and,
wherein the specialized travel policy is programmatically associated with the first participant dataset.

6. The system of claim 5, wherein the clinical dataset further comprises a second participant dataset;
wherein the GUI further comprises a participant travel policies interface having a participant policy control;
wherein the participant travel policies interface receives from the database and displays the second participant dataset;
wherein the GUI is configured to enable using participant policy control to associate the second participant dataset with the second travel policy condition; and,
wherein responsive to using the using participant policy control the GUI is configured to (a) cause the database to programmatically associate the second travel policy condition with the second participant dataset and (b) cause the database to programmatically associate the new specialized travel policy with the second participant dataset.

7. The system of claim 6 wherein the second travel policy condition is selected from the country, the clinical site, and the traveler policy label; and,
wherein the new specialized travel policy is selected from the country policy, the site policy, and the custom policy.

8. The system of claim 2 wherein the clinical study dataset further comprises a first visit and a second visit;
wherein the first visit comprises a first visit attribute;
wherein the second visit comprises the visit policy label;
wherein the travel policy data set comprises:
the first visit policy associated with the first visit attribute; and,
the second visit policy associated with the visit policy label;
wherein the first visit policy is programmatically associated with the first visit;
wherein the second visit policy is programmatically associated with the second visit;
wherein the GUI further comprises a visit schedule control;
wherein the GUI is configured to enable using of the visit schedule control to add a third visit having the first visit attribute; and,
wherein responsive to using of the visit schedule control to add the third visit the GUI is configured to cause the database to programmatically associate the third visit with the first visit policy.

9. The system of claim 8, wherein the clinical study dataset further comprises a fourth visit;
wherein the GUI is configured to enable using the visit schedule control to associate the fourth visit with the visit policy label; and,
wherein responsive to using the visit schedule control to associate the fourth visit with the visit policy label the GUI is configured to cause the database to programmatically associate the fourth visit with the second visit policy.
